(19) United States
(12) Patent Application Publication
(10) Pub. No.: US 2008/0226708 A1
(43) Pub. Date: Sep. 18, 2008

(54) Silicone Vesicles Containing Actives

(76) Inventors: Shaow Burn Lin, Midland, MI (US); James Thompson, Sanford, MI (US); Joanna Newton, Braine-L'Alleud (BE); Stephanie Postiaux, le Roeulx (BE)

Correspondence Address: DOW CORNING CORPORATION CO1232, 2200 W. SALZBURG ROAD, P.O. BOX 994, MIDLAND, MI, 48686-0994, US

(21) Appl. No.: 12/091,185

(22) PCT Filed: Oct. 25, 2006
(86) PCT No.: PCT/US06/41831

§ 371 Date: Apr. 23, 2008

Related U.S. Application Data
(60) Provisional application No. 60/732,392, filed on Nov. 1, 2005.
(60) Provisional application No. 60/732,841, filed on Nov. 2, 2005.

(51) Int. Cl. A61K 9/127; A61K 8/89; A61Q 5/00; A61Q 1/00; A61Q 3/00; A61K 8/18

(52) U.S. Cl. 424/450; 424/70.12; 424/65; 424/64; 424/63; 424/59; 424/61; 510/122; 510/159

(57) ABSTRACT

Processes are disclosed for preparing silicone vesicle compositions and emulsions containing silicone vesicles, the compositions prepared therefrom, and formulated personal and healthcare products containing the silicone vesicles and emulsions compositions.

### CROSS REFERENCE TO RELATED APPLICATIONS

This application claims priority to U.S. Application No. 60/732,392, filed 1 Nov., 2005, and U.S. Application No. 60/732,841, filed 2 Nov., 2006.

### TECHNICAL FIELD

This invention relates to processes for preparing silicone vesicle compositions and emulsions containing silicone vesicles, the compositions prepared therefrom, and formulated personal and healthcare products containing the silicone vesicles and emulsions compositions.

### BACKGROUND

Long-standing needs in the field of cosmetic and drug formulation/delivery field are to identify vesicle compositions that form and entrap actives easily, are stable under various chemical and mechanical stresses, and yet are able to deliver the actives in a controlled manner under desired conditions. Vesicles derived from silicone surfactants, and more particularly silicone polyether surfactants, are of interest because of additional inherent benefits that this class of surfactants possesses vs. other types. For example, silicone polyether surfactants often have improved aesthetics in personal care formulations.

A long standing need in the field of vesicles is the simultaneous entrapment of hydrophobic and hydrophilic actives. Often, vesicle structures are optimized to entrap either a hydrophobic or hydrophilic active. It has proven to be difficult to simultaneously entrap hydrophobic and hydrophilic actives efficiently in the same vesicle composition that remain stable with time.

The present inventors have found a efficient process that simultaneously entraps both hydrophobic and hydrophilic actives using silicone based vesicles.

### SUMMARY

This invention relates to a process for preparing a vesicle composition comprising:

I) combining;
A) an organopolysiloxane having at least one hydrophilic substituent group,
B) a water miscible solvent,
D′) a hydrophilic active,
   to form a dispersion of the hydrophilic active,

II) combining;
A) an organopolysiloxane having at least one hydrophilic substituent group,
C) optionally, a silicone or organic oil,
D″) a hydrophobic active,
   to form a dispersion of the hydrophobic active,

III) combining the dispersion of the hydrophilic active and the dispersion of the hydrophobic active and admixing water to form vesicles.

This invention also relates to a process for preparing a emulsion containing vesicles comprising;

I) combining,
A) an organopolysiloxane having at least one hydrophilic substituent group,
B) a water miscible volatile solvent,
C) optionally, a silicone or organic oil,
D) a personal care or health care active,
   with water to form an aqueous dispersion,

II) mixing the aqueous dispersion to form vesicles,

III) optionally, removing the water miscible volatile solvent from the vesicles,

IV) adding the vesicles to an emulsion.

The present invention further relates to vesicle and emulsion compositions prepared by the inventive process, and to personal, household, health care product compositions containing the vesicle compositions.

### DETAILED DESCRIPTION

### A) Organopolysiloxane Component

Component A) is an organopolysiloxane having at least one hydrophilic substituent group. Organopolysiloxanes are well known in the art and are often designated as comprising any number of “M” siloxy units (R₃SiO_{0.5}), “D” siloxy units (R₂SiO), “T” siloxy units (RSiO_{1.5}), or “Q” siloxy units (SiO₂) where R is independently any hydrocarbon group. In the present invention, the organopolysiloxane has at least hydrophilic substituent. That is, at least one of the R hydrocarbon groups present in the organopolysiloxane is a hydrophilic group. For purposes of this invention, “hydrophilic group” is the accepted meaning in the art, i.e. designating water loving chemical moieties. Thus, the hydrophilic group can be selected from various cationic, anionic, zwitterionic, polyoxyalkylene, oxoazoline chemical moieties that are commonly used in combination with various hydrophobic chemical moieties to create surfactant structures or molecules having surface-active behavior.

The amount of the hydrophilic substituent on the organopolysiloxane can vary, depending on the specific chemical component, providing there is at least one hydrophilic group present on the organopolysiloxane. However, the amount of the hydrophilic groups present in the organopolysiloxane can be described by its weight percent, or in particular, the weight percent of the organopolysiloxane and weight percent of the total hydrophilic groups present in the molecule. Typically, the weight percent of the siloxane units in the organopolysiloxane can vary from 20 to 85, alternatively from 30 to 85, or alternatively from 35 to 80 weight percent, while the remaining weight portion of the organopolysiloxane is the hydrophilic group.

In one embodiment of the present invention, the organopolysiloxane having at least one hydrophilic substituent group is selected from silicone polyethers. Silicone polyethers (SPEs) generally refer to silicones containing polyether or polyoxyalkylene groups, which could take in many different structural forms. One such form is rake-type SPEs which are derived most commonly from hydrosilylation of SiH functional organosiloxanes with allyloxy-functional polyethers in the presence of a Pt catalyst. In this embodiment, component (A) is a silicone polyether having the structure represented by:

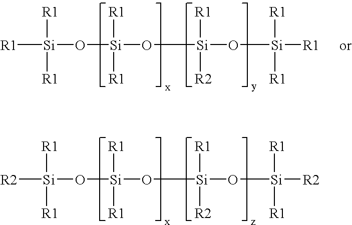

In these structures, R1 represents an alkyl group containing 1-6 carbon atoms such as methyl, ethyl, propyl, butyl, pentyl, and hexyl; R2 represents the group —(CH₂)ₐO(C₂H₄O)_{b}(C₃H₆O)_{c}R3; x has a value of 1-1,000, alternatively 1-500, or alternatively 10-300; y has a value of 1-500, alternatively 1-100, or alternatively 2-50; z has a value of 1-500, or alternatively 1-100; a has a value of 3-6; b has a value of 4-20; c has a value of 0-5; and R3 is hydrogen, a methyl group, or an acyl group such as acetyl. Typically, R1 is methyl; b is 6-12; c is zero; and R3 is hydrogen.

Preferably, the rake type SPE the silicone polyether has a D/D′ ratio (i.e. x/y ratio) ranging from 5/1 to 50/1, alternatively from 15/1 to 50/1 or alternatively from 20/1 to 50/1.

In a second embodiment, component (A) is an (AB)ₙ block silicone polyether (polyorganosiloxane-polyoxyalkylene block copolymer) having the average formula;

—[R¹(R₂SiO)_{x′}(R₂SiR¹O)(CₘH₂ₘO)_{y′}]ₙ— [Formula ]

where x and y′ are greater than 4, m is from 2 to 4 inclusive, n is greater than 2, R is independently a monovalent organic group containing 1 to 20 carbons, R¹ is a divalent hydrocarbon containing 2 to 30 carbons.

The siloxane block in Formula I is a predominately linear siloxane polymer having the formula (R₂SiO)_{x′}, wherein R is independently selected from a monovalent organic group, x′ is a integer greater than 4, alternatively x′ ranges from 20 to 100, or from 30 to 75.

The organic groups represented by R in the siloxane polymer are free of aliphatic unsaturation. These organic groups may be independently selected from monovalent hydrocarbon and monovalent halogenated hydrocarbon groups free of aliphatic unsaturation. These monovalent groups may have from 1 to 20 carbon atoms, alternatively 1 to 10 carbon atoms, and are exemplified by, but not limited to alkyl groups such as methyl, ethyl, propyl, butyl, pentyl, hexyl, heptyl, octyl, undecyl, and octadecyl; cycloalkyl such as cyclohexyl; aryl such as phenyl, tolyl, xylyl, benzyl, and 2-phenylethyl; and halogenated hydrocarbon groups such as 3,3,3-trifluoropropyl, 3-chloropropyl, and dichlorophenyl. At least 50 percent, alternatively at least 80%, of the organic groups in the organopolysiloxane may be methyl (denoted as Me). Typically, the siloxane block is a predominately linear polydimethylsiloxane having the formula (Me₂SiO)_{x′}, where x′ is as defined above.

The polyoxyalkylene block of the silicone polyether is represented by the formula (CₘH₂ₘO)_{y′} wherein m is from 2 to 4 inclusive, and y′ is greater than 4, alternatively y′ can range from 5 to 30, or alternatively from 5 to 22. The polyoxyalkylene block typically can comprise oxyethylene units (C₂H₄O)_{y′}, oxypropylene units (C₃H₆O)_{y′}, oxybutylene units (C₄H₈O)_{y′}, or mixtures thereof. Typically, the polyoxyalkylene block comprises oxyethylene units (C₂H₄O)_{y′}.

At least one end of each polyoxyalkylene block in Formula I is linked to a siloxane block by a divalent organic group, designated R¹. This linkage is determined by the reaction employed to prepare the (AB)ₙ block silicone polyether copolymer. The divalent organic groups of R¹ may be independently selected from divalent hydrocarbons containing 2 to 30 carbons and divalent organofunctional hydrocarbons containing 2 to 30 carbons. Representative, non-limiting examples of such divalent hydrocarbon groups include; ethylene, propylene, butylene, pentylene, hexylene, heptylene, octylene, and the like. Representative, non-limiting examples of such divalent organofunctional hydrocarbons groups include acrylate and methacrylate. Typically, R¹ is propylene, (—CH₂CH₂CH₂—).

The (AB)ₙ block silicone polyethers are endblocked. The endblocking unit is also determined by the reaction employed to prepare the (AB)ₙ block silicone polyether copolymer, which is generally the residual reactive groups of the reactants used. For example, the (AB)ₙ block silicone polyether copolymers can be prepared by the metal catalyzed hydrosilylation reaction of a diallyl polyether (i.e. an allyl group is present on each molecular terminal end) with a SiH terminated polyorganosiloxane. The resulting (AB)ₙ block silicone polyether copolymer would have polyoxyalkylene blocks linked to the silicone blocks via a propyleneoxy group (—CH₂CH₂CH₂O—), and using a slight molar excess of the allyl polyether would result in an allyl endblock unit (—CH₂CHCH₂). Alternative endblock units can result from the addition of other molecules in the reaction employed to prepare the (AB)ₙ block silicone polyether copolymer that are capable of reacting with the siloxane or polyether block intermediates. For example, the addition of organic compounds having mono-terminated aliphatic unsaturation (such as a mono allyl terminated polyether) will result in the endcapping of the (AB)ₙ block silicone polyether copolymer with that organic compound. Preferably, the endblocking units of the (AB)ₙ block silicone polyether is an allyl ether (CH₂═CHCH₂O—), an allyl polyether, a methylallyl ether, or a methylallyl polyether.

The molecular weights of the (AB)ₙ block silicone polyether copolymers will be determined by the number of repeating siloxane and polyoxyethylene blocks, as indicated by the subscript n in Formula I. Typically, the value of n is such to provide weight average molecular weights (M_{W}) to range from 1,500 to 150,000, alternatively, from 10,000 to 100,000.

The (AB)ₙ SPEs of the present vesicle compositions have a molar ratio of the total siloxane units to the polyoxyethylene units in the (AB)ₙ block silicone polyether. This molecular parameter is expressed by the value of x′/(x′+y′) in Formula I. The value of x′/(x′+y′) can vary from 0.4 to 0.9, or alternatively from 0.55 to 0.9.

The (AB)ₙ SPEs useful to prepare the vesicle compositions of the present invention can be prepared by any method known in the art for preparing such block copolymers. Typically however, the (AB)ₙ SPEs useful in the preparation of the vesicle compositions of the present invention are obtained from a method comprising reacting an SiH terminated organopolysiloxane with a polyoxyethylene having an unsaturated hydrocarbon group at each molecular terminal, in a hydrosilylation reaction, wherein the mole ratio of the unsaturated hydrocarbon groups to SiH in the reaction is at least 1:1.

### B) Water-Miscible Solvent

Component B) is a water-miscible volatile solvent. As used herein “water-miscible” means the solvent forms a dispersion with water at room temperature for at least several hours. Component B′) is a water-miscible volatile solvent. “Volatile” means the solvent has a higher vapor pressure than water at various temperatures. As such, when the aqueous dispersion of the organopolysiloxane and solvent are subjected to conditions to remove the solvent, such as heating the dispersion under reduced pressures, the volatile solvent is primarily removed first, allowing all or most of the water to remain in the composition.

Suitable water-miscible volatile solvents for vesicle dispersion preparation include organic solvents such as alcohols, ethers, glycols, esters, acids, halogenated hydrocarbons, diols. The organic solvents should be miscible with water at the proportion and lower in order to effectively disperse silicones and maintain stable and uniform dispersion overtime. For the purpose of illustration, water-miscible alcohols include method, ethanol, propanol, isopropanol, butanol, and higher hydrocarbon alcohols; ethers include gylcol ethers, methyl-ethyl ether, methyl isobutyl ether (MIBK), etc; glycols include propylene glycols, esters include esters of triglycerol, the esterification products of acid and alcohol; halogenated hydrocarbons include chloroform. Typically water-miscible organic solvents are solvents with relatively low boiling points (<10° C.) or high evaporation rate, so they may be removed under vacuum with ease. The most preferred water-miscible organic solvents for this invention are volatile alcohols including methanol, ethanol, isopropanol, and propanol. These alcohols can be removed from aqueous mixtures containing silicone vesicle dispersions via vacuum stripping at ambient temperature.

### C) Optional Silicone or Organic Oil Component

Optional component C) is a silicone or organic oil. The silicone can be any organopolysiloxane having the general formula RᵢSiO_{(4-i)/2} in which i has an average value of one to three and R is a monovalent organic group. The organopolysiloxane can be cyclic, linear, branched, and mixtures thereof.

In one embodiment, component C) is a volatile methyl siloxane (VMS) which includes low molecular weight linear and cyclic volatile methyl siloxanes. Volatile methyl siloxanes conforming to the CTFA definition of cyclomethicones are considered to be within the definition of low molecular weight siloxane.

Linear VMS have the formula (CH₃)₃SiO{(CH₃)₂SiO}_{f}Si(CH₃)₃. The value of f is 0-7. Cyclic VMS have the formula {(CH₃)₂SiO}_{g}. The value of g is 3-6. Preferably, these volatile methyl siloxanes have a molecular weight of less than 1,000; a boiling point less than 250° C.; and a viscosity of 0.65 to 5.0 centistoke (mm²/s), generally not greater than 5.0 centistoke (mm²/s).

Representative linear volatile methyl siloxanes are hexamethyldisiloxane (MM) with a boiling point of 100° C., viscosity of 0.65 mm²/s, and formula Me₃SiOSiMe₃; octamethyltrisiloxane (MDM) with a boiling point of 152° C., viscosity of 1.04 mm²/s, and formula Me₃SiOMe₂SiOSiMe₃; decamethyltetrasiloxane (MD₂M) with a boiling point of 194° C., viscosity of 1.53 mm²/s, and formula Me₃SiO(Me₂SiO)₂SiMe₃; dodecamethylpentasiloxane (MD₃M) with a boiling point of 229° C., viscosity of 2.06 mm²/s, and formula Me₃SiO(Me₂SiO)₃SiMe₃; tetradecamethylhexasiloxane (MD₄M) with a boiling point of 245° C., viscosity of 2.63 mm²/s, and formula Me₃SiO(Me₂SiO)₄SiMe₃; and hexadecamethylheptasiloxane (MD₅M) with a boiling point of 270° C., viscosity of 3.24 mm²/s, and formula Me₃SiO(Me₂SiO)₅SiMe₃.

Representative cyclic volatile methyl siloxanes are hexamethylcyclotrisiloxane (D₃), a solid with a boiling point of 134° C., a molecular weight of 223, and formula {(Me₂)SiO}₃; octamethylcyclotetrasiloxane (D₄) with a boiling point of 176° C., viscosity of 2.3 mm²/s, a molecular weight of 297, and formula {(Me₂)SiO}₄; decamethylcyclopentasiloxane (D₅) with a boiling point of 210° C., viscosity of 3.87 mm²/s, a molecular weight of 371, and formula {(Me₂)SiO}₅; and dodecamethylcyclohexasiloxane (D₆) with a boiling point of 245° C., viscosity of 6.62 mm²/s, a molecular weight of 445, and formula {(Me₂)SiO}₆.

The silicone selected as component C) can be any polydiorganosiloxane fluid, gum, or mixtures thereof. If the polyorganosiloxane has a molecular weight equal to or greater than 1000, it can be blended with the volatile methyl siloxanes described above. The polydiorganosiloxane gums suitable for the present invention are essentially composed of dimethylsiloxane units with the other units being represented by monomethylsiloxane, trimethylsiloxane, methylvinylsiloxane, methylethylsiloxane, diethylsiloxane, methylphenylsiloxane, diphenylsiloxane, ethylphenylsiloxane, vinylethylsiloxane, phenylvinylsiloxane, 3,3,3-trifluoropropylmethylsiloxane, dimethylphenylsiloxane, methylphenylvinylsiloxane, dimethylethylsiloxane, 3,3,3-trifluoropropyldimethylsiloxane, mono-3,3,3-trifluoropropylsiloxane, aminoalkylsiloxane, monophenylsiloxane, monovinylsiloxane and the like.

When component C) is an organic oil, it may be selected from any organic oil known in the art suitable for use in the preparation of personal, household, or healthcare formulations. Suitable organic oils include, but are not limited to, natural oils such as coconut oil; hydrocarbons such as mineral oil and hydrogenated polyisobutene; fatty alcohols such as octyldodecanol; esters such as C12-C15 alkyl benzoate; diesters such as propylene dipelarganate; and triesters, such as glyceryl trioctanoate. The organic oil components can also be mixture of low viscosity and high viscosity oils. Suitable low viscosity oils have a viscosity of 5 to 100 mPa·s at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or mixtures of octyldodecanol, acetylated lanolin alcohol, cetyl acetate, isododecanol, polyglyceryl-3-diisostearate, or mixtures thereof. The high viscosity surface oils generally have a viscosity of 200-1,000,000 mPa·s at 25° C., preferably a viscosity of 100,000-250,000 mPa·s. Surface oils include castor oil, lanolin and lanolin derivatives, triisocetyl citrate, sorbitan sesquioleate, C10-18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or mixtures thereof. Mention may be made, among the optional other non-silicone fatty substances, of mineral oils, such as liquid paraffin or liquid petroleum, of animal oils, such as perhydrosqualene or arara oil, or alternatively of vegetable oils, such as sweet almond, calophyllum, palm, castor, avocado, jojaba, olive or cereal germ oil. It is also possible to use esters of lanolic acid, of oleic acid, of lauric acid, of stearic acid or of myristic acid, for example; alcohols, such as oleyl alcohol, linoleyl or linolenyl alcohol, isostearyl alcohol or octyldodecanol; or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

These organic oils have some effect to improve the solubility of D) component, personal or healthcare active substances in vesicle system, stimulate transdermal absorption of these active substances, give better water-proofness and touch to the product. Some esters have notable effect to stimulate transdermal absorption of D) component. Some waxes have the advantage that they improve the stability, decay resistances, water-proofness and retention of personal or healthcare active substances in vesicle system of the product. Further, the product would be kind to skin and have an affable image to consumers by confining these organic oils to those from natural plants or seaweeds extract, i.e. olive oil or sweet almond oil.

### D) Hydrophobic or Hydrophilic Active

Component D) is either a hydrophilic (D′) or hydrophobic (D″) active. The active may be selected from any personal or health care active. As used herein, a “personal care active” means any compound or mixtures of compounds that are known in the art as additives in the personal care formulations that are typically added for the purpose of treating hair or skin to provide a cosmetic and/or aesthetic benefit. A “healthcare active” means any compound or mixtures of compounds that are known in the art to provide a pharmaceutical or medical benefit. Thus, “healthcare active” include materials consider as an active ingredient or active drug ingredient as generally used and defined by the United States Department of Health & Human Services Food and Drug Administration, contained in Title 21, Chapter I, of the Code of Federal Regulations, Parts 200-299 and Parts 300-499.

Thus, active ingredient can include any component that is intended to furnish pharmacological activity or other direct effect in the diagnosis, cure, mitigation, treatment, or prevention of disease, or to affect the structure or any function of the body of a human or other animals. The phrase can include those components that may undergo chemical change in the manufacture of drug products and be present in drug products in a modified form intended to furnish the specified activity or effect.

Some representative examples of active ingredients include; drugs, vitamins, minerals; hormones; topical antimicrobial agents such as antibiotic active ingredients, antifungal active ingredients for the treatment of athlete's foot, jock itch, or ringworm, and acne active ingredients; astringent active ingredients; deodorant active ingredients; wart remover active ingredients; corn and callus remover active ingredients; pediculicide active ingredients for the treatment of head, pubic (crab), and body lice; active ingredients for the control of dandruff, seborrheic dermatitis, or psoriasis; and sunburn prevention and treatment agents.

By forming into silicone vesicles, these active ingredients are efficiently kept on the skin and result in a longer-lasting effect of the product. Further, we can control the stimulation or inhibition of transdermal absorption of active ingredients by formulating some additives. For example, some active ingredients are efficiently absorbed through the skin by formulating ethanol as volatile content, esters or menthol as stimulator of transdermal absorption. Especially, combination of aqueous active ingredients with silicone vesicles containing oil-soluble ones have advantages to stimulate transdermal absorption of these ingredients.

Useful active ingredients for use in processes according to the invention include vitamins and its derivatives, including “pro-vitamins”. Vitamins useful herein include, but are not limited to, Vitamin A₁, retinol, C₂-C₁₈ esters of retinol, vitamin E, tocopherol, esters of vitamin E, and mixtures thereof. Retinol includes trans-retinol, 1,3-cis-retinol, 11-cis-retinol, 9-cis-retinol, and 3,4-didehydro-retinol, Vitamin C and its derivatives, Vitamin B₁, Vitamin B₂, Pro Vitamin B5, panthenol, Vitamin B₆, Vitamin B₁₂, niacin, folic acid, biotin, and pantothenic acid. Other suitable vitamins and the INCI names for the vitamins considered included herein are ascorbyl dipalmitate, ascorbyl methylsilanol pectinate, ascorbyl palmitate, ascorbyl stearate, ascorbyl glucocide, sodium ascorbyl phosphate, sodium ascorbate, disodium ascorbyl sulfate, potassium (ascorbyl/tocopheryl) phosphate.

RETINOL, it should be noted, is an International Nomenclature Cosmetic Ingredient Name (INCI) designated by The Cosmetic, Toiletry, and Fragrance Association (CTFA), Washington D.C., for vitamin A. Other suitable vitamins and the INCI names for the vitamins considered included herein are RETINYL ACETATE, RETINYL PALMITATE, RETINYL PROPIONATE, α-TOCOPHEROL, TOCOPHERSOLAN, TOCOPHERYL ACETATE, TOCOPHERYL LINOLEATE, TOCOPHERYL NICOTINATE, and TOCOPHERYL SUCCINATE.

Some examples of commercially available products suitable for use herein are Vitamin A Acetate and Vitamin C, both products of Fluka Chemie AG, Buchs, Switzerland; COVI-OX T-50, a vitamin E product of Henkel Corporation, La Grange, Ill.; COVI-OX T-70, another vitamin E product of Henkel Corporation, La Grange, Ill.; and vitamin E Acetate, a product of Roche Vitamins & Fine Chemicals, Nutley, N.J.

The active ingredient used in processes according to the invention can be an active drug ingredient. Representative examples of some suitable active drug ingredients which can be used are hydrocortisone, ketoprofen, timolol, pilocarpine, adriamycin, mitomycin C, morphine, hydromorphone, diltiazem, theophylline, doxorubicin, daunorubicin, heparin, penicillin G, carbenicillin, cephalothin, cefoxitin, cefotaxime, 5-fluorouracil, cytarabine, 6-azauridine, 6-thioguanine, vinblastine, vincristine, bleomycin sulfate, aurothioglucose, suramin, mebendazole, clonidine, scopolamine, propranolol, phenylpropanolamine hydrochloride, ouabain, atropine, haloperidol, isosorbide, nitroglycerin, ibuprofen, ubiquinones, indomethacin, prostaglandins, naproxen, salbutamol, guanabenz, labetalol, pheniramine, metrifonate, and steroids.

Considered to be included herein as active drug ingredients for purposes of the present invention are antiacne agents such as benzoyl peroxide and tretinoin; antibacterial agents such as chlorohexadiene gluconate; antifungal agents such as miconazole nitrate; anti-inflammatory agents; corticosteroidal drugs; non-steroidal anti-inflammatory agents such as diclofenac; antipsoriasis agents such as clobetasol propionate; anesthetic agents such as lidocaine; antipruritic agents; antidermatitis agents; and agents generally considered barrier films.

The active component D) of the present invention can be a protein, such as an enzyme. The internal inclusion of enzymes in the silicone vesicle have advantages to prevent enzymes from deactivating and maintain bioactive effects of enzymes for longer time. Enzymes include, but are not limited to, commercially available types, improved types, recombinant types, wild types, variants not found in nature, and mixtures thereof. For example, suitable enzymes include hydrolases, cutinases, oxidases, transferases, reductases, hemicellulases, esterases, isomerases, pectinases, lactases, peroxidases, laccases, catalases, and mixtures thereof. Hydrolases include, but are not limited to, proteases (bacterial, fungal, acid, neutral or alkaline), amylases (alpha or beta), lipases, mannanases, cellulases, collagenases, lisozymes, superoxide dismutase, catalase, and mixtures thereof. Said protease include, but are not limited to, trypsin, chymotrypsin, pepsin, pancreatin and other mammalian enzymes; papain, bromelain and other botanical enzymes; subtilisin, epidermin, nisin, naringinase(L-rhammnosidase) urokinase and other bacterial enzymes. Said lipase include, but are not limited to, triacyl-glycerol lipases, monoacyl-glycerol lipases, lipoprotein lipases, e.g. steapsin, erepsin, pepsin, other mammalian, botanical, bacterial lipases and purified ones. Natural papain is preferred as said enzyme. Further, stimulating hormones, e.g. insulin, can be used together with these enzymes to boost the effectiveness of them.

Component D) may also be a sunscreen agent. The sunscreen agent can be selected from any sunscreen agent known in the art to protect skin from the harmful effects of exposure to sunlight. The sunscreen compound is typically chosen from an organic compound, an inorganic compound, or mixtures thereof that absorbs ultraviolet (UV) light. Thus, representative non limiting examples that can be used as the sunscreen agent include; Aminobenzoic Acid, Cinoxate, Diethanolamine Methoxycinnamate, Digalloyl Trioleate, Dioxybenzone, Ethyl 4-[bis(Hydroxypropyl)]Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate O, Phenylbenzimidazole Sulfonic Acid, Red Petrolatum, Sulisobenzone, Titanium Dioxide, and Trolamine Salicylate, cetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Ceria/Silica, Ceria/Silica Talc, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphtliopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, Zinc Dioxide, Serium Dioxide, TriPABA Panthenol, Urocanic Acid, and VA/Crotonates/Methacryloxybenzophenone-1 Copolymer.

These sunscreen agent can be selected one or combination of more than one. Further, the silicone vesicle can contain one sunscreen agent in inner phase, and another in outer phase, e.g. containing oil-soluble sunscreen agent in inner phase and water-dispersible one in outer phase of this silicone vesicle. In this usage, the silicone vesicle is useful to stabilize the combination of different sunscreens for some organic sunscreen agents are colored by contacting with Titanium dioxide directly.

Alternatively, the sunscreen agent is a cinnamate based organic compound, or alternatively, the sunscreen agent is octyl methoxycinnamate, such as Uvinul® MC 80 an ester of para-methoxycinnamic acid and 2-ethylhexanol.

Component D) may also be a fragrance or perfume. The perfume can be any perfume or fragrance active ingredient commonly used in the perfume industry. These compositions typically belong to a variety of chemical classes, as varied as alcohols, aldehydes, ketones, esters, ethers, acetates, nitrites, terpenic hydrocarbons, heterocyclic nitrogen or sulfur containing compounds, as well as essential oils of natural or synthetic origin. Many of these perfume ingredients are described in detail in standard textbook references such as Perfume and Flavour Chemicals, 1969, S. Arctander, Montclair, N.J.

Fragrances may be exemplified by, but not limited to, perfume ketones and perfume aldehydes. Illustrative of the perfume ketones are buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-lonone, Beta-lonone, Gamma-Methyl so-called lonone, Fleuramone, Dihydrojasmone, Cis-Jasmone, Iso-E-Super, Methyl-Cedrenyl-ketone or Methyl-Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)propyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbornane, 6,7-Dihydro-1,1,2,3,3-Pentamethyl-4(5H)-Indanone, 4-Damascol, Dulcinyl or Cassione, Gelsone, Hexylon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, and Tetrameran.

More preferably, the perfume ketones are selected for its odor character from Alpha Damascone, Delta Damascone, Iso Damascone, Carvone, Gamma-Methyl-lonone, Iso-E-Super, 2,4,4,7-Tetramethyl-oct-6-en-3-one, Benzyl Acetone, Beta Damascone, Damascenone, methyl dihydrojasmonate, methyl cedrylone, and mixtures thereof.

Preferably, the perfume aldehyde is selected for its odor character from adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-para-methoxyphenyl propanal, 2-methyl-4-(2,6,6-trimethyl-2(1)-cyclohexen-1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnanmaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 5 or 6 methoxy 10 hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, paratolylacetaldehyde; 4-methylphenylacetaldehyde, 2-methyl-4-(2,6,6-trimethyl 1-cyclohexen-1-yl)-2-butena 1, ortho-methoxycinnamic aldehyde, 3,5,6-trmethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl)benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, hexanal, trans-2-hexenal, 1-p-menthene-q-carboxaldehyde and mixtures thereof.

More preferred aldehydes are selected for their odor character from 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, hexanal, trans-2-hexenal, and mixture thereof.

In the above list of perfume ingredients, some are commercial names conventionally known to one skilled in the art, and also includes isomers. Such isomers are also suitable for use in the present invention.

Component D) may also be one or more plant extract. Examples of these components are as follows: Ashitaba extract, avocado extract, hydrangea extract, Althea extract, Arnica extract, aloe extract, apricot extract, apricot kernel extract, Ginkgo Biloba extract, fennel extract, turmeric[Curcuma] extract, oolong tea extract, rose fruit extract, Echinacea extract, Scutellaria root extract, Phellodendro bark extract, Japanese Coptis extract, Barley extract, Hyperium extract, White Nettle extract, Watercress extract, Orange extract, Dehydrated saltwater, seaweed extract, hydrolyzed elastin, hydrolyzed wheat powder, hydrolyzed silk, Chamomile extract, Carrot extract, Artemisia extract, Glycyrrhiza extract, hibiscustea extract, Pyracantha Fortuneana Fruit extract, Kiwi extract, Cinchona extract, cucumber extract, guanocine, Gardenia extract, Sasa Albo-marginata extract, Sophora root extract, Walnut extract, Grapefruit extract, Clematis extract, Chlorella extract, mulberry extract, Gentiana extract, black tea extract, yeast extract, burdock extract, rice bran ferment extract, rice germ oil, comfrey extract, collagen, cowberry extract, Gardenia extract, Asiasarum Root extract, Family of Bupleurum extract, umbilical cord extract, Salvia extract, Saponaria extract, Bamboo extract, Crataegus fruit extract, Zanthoxylum fruit extract, shiitake extract, Rehmannia root extract, gromwell extract, Perilla extract, linden extract, Filipendula extract, peony extract, Calamus Root extract, white birch extract, Horsetail extract, Hedera Helix (Ivy) extract, hawthorn extract, Sambucus nigra extract, Achillea millefolium extract, Mentha piperita extract, sage extract, mallow extract, Cnidium officinale Root extract, Japanese green gentian extract, soybean extract, jujube extract, thyme extract, tea extract, clove extract, Gramineae imperata cyrillo extract, Citrus unshiu peel extract Japanese Angellica Root extract, Calendula extract, Peach Kernel extract, Bitter orange peel extract, Houttuyna cordata extract, tomato extract, natto extract, Ginseng extract, Green tea extract (camelliea sinesis), garlic extract, wild rose extract, hibiscus extract, Ophiopogon tuber extarct, Nelumbo nucifera extract, parsley extract, honey, hamamelis extract, Parietaria extract, Isodonis herba extract, bisabolol extract, Loquat extract, coltsfoot extract, butterbur extract, Porid cocos wolf extract, extract of butcher's broom, grape extract, propolis extract, luffa extract, safflower extract, peppermint extract, linden tree extract, Paeonia extract, hop extract, pine tree extract, horse chestnut extract, Mizu-bashou [Lysichiton camtschatcese] extract, Mukurossi peel extract, Melissa extract, peach extract, cornflower extract, eucalyptus extract, saxifrage extract, citron extract, coix extract, mugwort extract, lavender extract, apple extract, lettuce extract, lemon extract, Chinese milk vetch extract, rose extract, rosemary extract, Roman Chamomile extract, and royal jelly extract.

The amount of components A), B), C), and D) can vary in the process, but typically range as follows;

A) 2 to 50 wt %, alternatively 2 to 25 wt %, or alternatively 1 to 15 wt %,

B) 1 to 50 wt %, alternatively 2 to 25 wt %, or alternatively 2 to 15 wt %,

C) 0 to 50 wt %, alternatively 1 to 20 wt %, or alternatively 2 to 10 wt %,

D) 0.05 to 20 wt %, alternatively 0.1 to 15 wt %, or alternatively 0.1 to 10 wt %,

and sufficient amount of water to provide the sum of the wt % of A), B), and C) and water content to equal 100%.

### Preparation of Vesicles

Step I in the process to prepare the vesicle composition of the present invention comprises;

I) combining;
A) an organopolysiloxane having at least one hydrophilic substituent group,
B) a water miscible solvent,
D′) a hydrophilic active,
   to form a dispersion of the hydrophilic active,
      Components A), B), and D′), as described above, can be combined in any order to form the dispersion of the hydrophilic active. Alternatively, components B) and D′) are first combined and then added to component A). Alternatively, components B), B′) and D′) are first combined, and then added to component A). Typically components A), B) or B′) when used, and D′) are combined using common mixing techniques. There are no special requirements or conditions needed to effect the mixing and formation of the dispersion of the hydrophilic active. Common stirring techniques are usually sufficient. Typically, sufficient stirring is applied to provide a homogeneous dispersion.

Step II in the process to prepare the vesicle composition of the present invention comprises;

II) combining;
A) an organopolysiloxane having at least one hydrophilic substituent group,
C) optionally, a silicone or organic oil,
D″) a hydrophobic active,
   to form a dispersion of the hydrophobic active.
      Components A), optionally C), and D′), as described above, can be combined in any order to form the dispersion of the hydrophobic active. Alternatively, components C) and D″) are first combined and then added to component A). Alternatively, components C), B′) and D″) are first combined, and then added to component A). Typically components A), C) or B′) when used, and D″) are combined using common mixing techniques. There are no special requirements or conditions needed to effect the mixing and formation of the dispersion of the hydrophobic active. Common stirring techniques are usually sufficient. Typically, sufficient stirring is applied to provide a homogeneous dispersion.

Steps I and II can occur in any order, that is, they do not need to be performed in sequential order.

Step III in the process to prepare the vesicle composition of the present invention comprises;

III) combining the dispersion of the hydrophilic active and the dispersion of the hydrophobic active and admixing water to form vesicles.

There are no special requirements or conditions needed to effect the mixing and formation of vesicles. Mixing techniques can be simple stirring, homogenizing, sonalating, and other mixing techniques known in the art to effect the formation of vesicles in aqueous dispersions. The mixing can be conducted in a batch, semi-continuous, or continuous process.

The formation of vesicles can be confirmed by techniques common in the state of the art. Typically, vesicles have a lamellar phase structure which exhibit birefringence when examined with a cross polarizing microscope. Alternatively, the formation of vesicles can be demonstrated by Cyro-Transmission Electron Microscopy (Cryo-TEM) techniques. Particle size measurements can also be used to indicate that the organopolysiloxanes are sufficiently dispersed in aqueous medium typical of vesicle sizes. For example, average particle sizes of less than 0.500 μm (micrometers), are typical for dispersed vesicles. Vesicles having an average particle size of less than 0.200 μm, or 0.100 μm are possible with the teachings of the present invention.

Step IV in the process of the present invention is optional, and involves removing the water miscible volatile solvent, component B′). Typically, the water miscible volatile solvent is removed by known techniques in the art, such as subjecting the vesicle composition to reduced pressures, while optionally heating the composition. Devices illustrative of such techniques include rotary evaporators and thin film strippers.

The present invention further encompasses the vesicle compositions prepared by the process described herein.

### Process for Preparing an Emulsion Containing Vesicles

This invention also relates to a process for preparing an emulsion containing vesicles comprising;

I) combining,
A) an organopolysiloxane having at least one hydrophilic substituent group,
B) a water miscible volatile solvent,
C) optionally, a silicone or organic oil,
D) a personal care or health care active,
   with water to form an aqueous dispersion,

II) mixing the aqueous dispersion to form vesicles,

III) optionally, removing the water miscible volatile solvent from the vesicles,

IV) adding the vesicles to an emulsion.

Components A), B), C), and D) are as described above. The amounts of components A), B), C), and D) used in the process to prepare the vesicle containing emulsions, but typically range as follows;

A) 2 to 50 wt %, alternatively 2 to 25 wt %, or alternatively 1 to 15 wt %,

B) 1 to 50 wt %, alternatively 2 to 25 wt %, or alternatively 2 to 15 wt %,

C) 0 to 50 wt %, alternatively 1 to 20 wt %, or alternatively 2 to 10 wt %,

D) 0.05 to 20 wt %, alternatively 0.1 to 15 wt %, or alternatively 0.1 to 10 wt %,

and sufficient amount of water to provide the sum of the wt % of A), B), and C) and water content to equal 100%.

The order of combining components A), B), C), and D) with water in the process for preparing emulsions containing vesicles is not critical, but typically A), B), C), and D) are first combined and then added with water to form an aqueous dispersions of components A)-D).

Step II in the process process for preparing emulsions containing vesicles is mixing the aqueous dispersion formed in Step I to form vesicles. There are no special requirements or conditions needed to effect the mixing and formation of vesicles. Mixing techniques can be simple stirring, homogenizing, sonalating, and other mixing techniques known in the art to effect the formation of vesicles in aqueous dispersions. The mixing can be conducted in a batch, semi-continuous, or continuous process.

The formation of vesicles can be confirmed by techniques common in the state of the art. Typically, vesicles have a lamellar phase structure which exhibit birefringence when examined with a cross polarizing microscope. Alternatively, the formation of vesicles can be demonstrated by Cyro-Transmission Electron Microscopy (Cryo-TEM) techniques. Particle size measurements can also be used to indicate that the organopolysiloxanes are sufficiently dispersed in aqueous medium typical of vesicle sizes. For example, average particle sizes of less than 0.500 μm (micrometers), are typical for dispersed vesicles. Vesicles having an average particle size of less than 0.200 μm, or 0.100 μm are possible with the teachings of the present invention.

Step III in the process for preparing emulsions containing vesicles is optional, and involves removing the water miscible volatile solvent, component B). Typically, the water miscible volatile solvent is removed by known techniques in the art, such as subjecting the vesicle composition to reduced pressures, while optionally heating the composition. Devices illustrative of such techniques include rotary evaporators and thin film strippers.

Step 1V) in the process for preparing emulsions containing vesicles involves adding the vesicles formed to an emulsion. As used herein, “emulsion” is meant to encompass water continuous emulsions (for example an oil in water type emulsion, or a silicone in water emulsion), oil or silicone continuous emulsions (water in oil emulsions or water in silicone emulsions), or multiple emulsions (water/oil/water, oil/water/oil types, water/silicone/water, or silicone/water/silicone). The vesicles formed may be added to any type of emulsion by common mixing techniques. There are no special requirements or conditions needed to effect the mixing of vesicles and the emulsion. Mixing techniques can be simple stirring, homogenizing, sonalating, and other mixing techniques known in the art to effect the formation of vesicles in aqueous dispersions. The mixing can be conducted in a batch, semi-continuous, or continuous process.

The amount of vesicles from step II) or step III) added to the emulsion in step IV) can vary and is not limited, however the amounts typically may range from a vesicle/emulsion weight ratio of 0.1/99 to 99/0.1, alternatively 1/99 to 99/1.

The emulsions used may be w/o, w/s, or multiple phase emulsions using silicone emulsifiers. Typically the water-in-silicone emulsifier in such formulation is non-ionic and is selected from polyoxyalkylene-substituted silicones, silicone alkanolamides, silicone esters and silicone glycosides. Silicone-based surfactants may be used to form such emulsions and are well known in the art, and have been described for example in U.S. Pat. No. 4,122,029 (Gee et al.), U.S. Pat. No. 5,387,417 (Rentsch), and U.S. Pat. No. 5,811,487 (Schulz et al).

When the emulsion is an oil-in-water emulsion, it may include common ingredients generally used for preparing emulsions such as but not limited to non ionic surfactants well known in the art to prepare o/w emulsions. Examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, and polyoxyalkylene glycol modified polysiloxane surfactants.

The vesicle and emulsion compositions prepared according to the invention can be used in various over-the-counter (OTC) personal care compositions, health care compositions, and household care compositions, but especially in the personal care arena. The vesicle and emulsion compositions prepared according to the present invention can be combined with a variety of personal, household, or healthcare ingredients in a formulated product composition. A listing of possible personal, household, or health care ingredients is taught in WO 03/101412, which is incorporated herein by reference. Thus, they can be used in antiperspirants, deodorants, skin creams, skin care lotions, moisturizers, facial treatments such as acne or wrinkle removers, personal and facial cleansers, bath oils, perfumes, colognes, sachets, sunscreens, pre-shave and after-shave lotions, liquid soaps, shaving soaps, shaving lathers, hair shampoos, hair conditioners, hair sprays, mousses, permanents, depilatories, hair cuticle coats, make-ups, color cosmetics, foundations, blushes, lipsticks, lip balms, eyeliners, mascaras, oil removers, color cosmetic removers, nail polishes, and powders.

The vesicle and emulsion compositions can be combined with a powder to provide a formulation base for a variety of cosmetic products. A powder is defined herein as a dry particulate matter having a particle size of 0.02-50 microns. The particulate matter may be colored or non-colored (for example white). Suitable powders include bismuth oxychloride, titanated mica, fumed silica, spherical silica beads, polymethylmethacrylate beads, micronized teflon, boron nitride, acrylate polymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium carbonate, magnesium hydroxide, magnesium oxide, magnesium silicate, magnesium trisilicate, maltodextrin, montmorillonite, microcrystalline cellulose, rice starch, silica, talc, mica, titanium dioxide, zinc laurate, zinc myristate, zinc neodecanoate, zinc rosinate, zinc stearate, polyethylene, alumina, attapulgite, calcium carbonate, calcium silicate, dextran, kaolin, nylon, silica silylate, silk powder, serecite, soy flour, tin oxide, titanium hydroxide, trimagnesium phosphate, walnut shell powder, or mixtures thereof, pearlpigments such as titanium oxide-coated mica, titanium oxide-coated mica, bismuth oxychloride, titanium oxide-coated bismuth oxychloride, titanium oxide-coated talc, fish scales, and titanium oxide-coated colored mica; metallic powder pigments such as aluminum powder, copper powder and stainless powder. The above mentioned powders may be surface treated with lecithin, amino acids, mineral oil, silicone oil, or various other agents either alone or in combination, which coat the powder surface and render the particles hydrophobic in nature.

The powder may also comprise various organic and inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Inorganic pigments generally consist of insoluble metallic salts of certified color additives, referred to as the Lakes or iron oxides. A pulverulent coloring agent, such as carbon black, chromium or iron oxides, ultramarines, manganese pyrophosphate, iron blue, and titanium dioxide, pearlescent agents, generally used as a mixture with colored pigments, or some organic dyes, generally used as a mixture with colored pigments and commonly used in the cosmetics industry, can be added to the composition. Tar pigments such as Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 505, Yellow No. 4, Yellow No. 5, Yellow No. 202, Yellow No. 203, Yellow No. 204, Yellow No. 401, Blue No. 1, Blue No. 2, Blue No. 201, Blue No. 404, Green No. 3, Green No. 201, Green No. 204, Green No. 205, Orange No. 201, Orange No. 203, Orange No. 204, Orange No. 206, and Orange No. 207; and natural pigments such as carminic acid, laccaic acid, carthamin, brazilin, and crocin, may be used. Pulverulent inorganic or organic fillers can also be added. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked. Mention may be made in particular of talc, mica, silica, kaolin, nylon powders (in particular ORGASOL), polyethylene powders, Teflon, starch, boron nitride, copolymer microspheres such as EXPANCEL (Nobel Industrie), POLYTRAP, and silicone resin microbeads (TOSPEARL from Toshiba, for example).

Further, these powders, pigments and fillers can be compounded or can be treated with common oil agents, silicone oil, fluorine-containing compounds and surfactants as far as the effect of the present invention is not prevented, as described above. For example, these powders may be or may not be surface-treated or modified in advance by, for example, the treatment with a fluorine-containing compound, treatment with a silicone resin, pendant treatment, treatment with a silane coupling agent, treatment with a titanium coupling agent, treatment with an oil agent, treatment with N-acylated lysine, treatment with a polyacrylic acid, treatment with a metal soap, treatment with an amino acid, treatment with an inorganic compound, plasma treatment, and mechanochemical treatment. If necessary, one or more kinds of surface treatments ormodification can be applied. According to the present invention, one or more kinds of the powders may be combined.

By covering these powders, pigments or fillers with the silicone vesicle, their touch and skin absorption can be improved, and help makeup last longer.

The silicone vesicle and emulsion compositions can be combined with humectants to provide a excellent moisture retention, feel to the touch on the skin or hair. The moisture retention effect of humectants is enhanced by the interaction of the organopolysiloxane forming vesicles and humectants. Suitable humectants include trehalose, pentaerythritol, xylitol, glycerin, propylene glycol, dipropylene glycol, tripropylene glycol, polypropyleneglycol, 1,3-butylene glycol, ethylene glycol, diethylene glycol, triethylene glycol, polyglycerin, hyaluronic acid and its salts, chondroitin sulfuric acid and its salts, pyrrolidone carboxylic acid salts, polyoxyethylene methylglucoside, polyoxypropylene methylglucoside, and ethylglucoside; sugar alcohols such as sorbitol, maltose, and maltitol; sterols such as cholesterol, sitosterol, phytosterol, and lanosterol; sugars and esters thereof; dextrin and derivatives thereof; panthenol and derivatives thereof and its salt; honey, urea, phospholipids, glucolipids, ceramides. Preferably, humectants can be selected from glycerin, diglycerin, propyleneglycol, 1,3-butylene glycol, polyethylene glycol, hyaturonic acid and its salts, glucolipids or ceramides. A desirable amount of the humectants to be added to the cosmetics ranges from 0.001 to 10 mass %, preferably 0.001 to 5 mass %, relative to the total formulation.

The silicone vesicle and emulsion compositions can be combined with thickening agents to prevent the destabilization of the cosmetics and silicone vesicle itself from adding other actives and components to the total formulation. Further, these thickening agents give the formulation “expensive” and good feel to touch. Preferably, thickening agents containing molecular frame of polyacrylic acids or polyacrylamide can be used for the intention. The following compounds are used as the thickening agent: plant-derived polymers such as gum Arabic, tragacanth gum, arabinogalactan, locust bean gum (carob gum), guar gum, karaya gum, carrageenan, pectin, agar-agar, quince seed (i.e., marmelo), starch from rice, corn, potato or wheat, algae colloid, and trant gum; bacteria-derived polymers such as xanthan gum, dextran, succinoglucan, and pullulan; animal-derived polymers such as collagen, casein, albumin, and gelatin; starch-derived polymers such as carboxymethyl starch and methylhydroxypropyl starch; cellulose polymers such as methyl cellulose, ethyl cellulose, methylhydroxypropyl cellulose, carboxymethyl cellulose, hydroxymethyl cellulose, hydroxypropyl cellulose, nitrocellulose, sodium cellulose sulfate, sodium carboxymethyl cellulose, crystalline cellulose, and cellulose powder; alginic acid-derived polymers such as sodium alginate and propylene glycol alginate; vinyl polymers such as polyvinyl methylether, polyvinylpyrrolidone, and carboxyvinyl polymer; polyoxyethylene polymers such as polyethylene glycol; polyoxyethylene/polyoxypropylene copolymers; acrylic polymers such as sodium polyacrylate, polyethyl acrylate, and polyacrylamide; polyethyleneimine; cationic polymers; and inorganic thickening agents such as, bentonite, aluminum magnesium silicate, laponite, smectite, saponite, hectorite, and silicic anhydride. The preferred thickening agent is silicone-based polyamides. Such polyamides are disclosed in WO99006473 (Barr et. al)

An oil-soluble gelling agent may also be used as the thickening agent. For example, at least one may be selected from the following group: metal soaps such as aluminum stearate, magnesium stearate, and zinc myristate; a amino acid derivatives such as N— lauroyl-L-glutamic acid, α,γ-di-n-butylamine; dextrin fatty acid esters such as dextrin palmitate, dextrin stearate, and dextrin 2-ethylhexane palmitate; sucrose fatty acid esters such as sucrose palmitate and sucrose stearate; benzylidene derivatives of sorbitol such as monobenzylidene sorbitol and dibenzylidene sorbitol; clay minerals modified with an organic moiety such as dimethylbenzyldodecylammonium montmorillonite clay, dimethyldioctadecylammonium montmorillonite, and octadecyldimethylbenzylammonium montmorillonite. A desirable amount of the thickening agent in the silicone vesicle ranges from 0.01 to 95 mass %, preferably from 0.1 to 50 mass %, relative to the total formulation.

For the formulation of hair dyes or other makeup products, coloring agents can be combined with the silicone vesicles. The silicone vesicles is covering the coloring agents on skin or hair to provide a stable coloring and reduce direct adhesion of coloring agents on skin or hair. These effect contribute to keep-well and low physiological load formulation. The coloring agents in the use of this invention can be selected from cationic or anionic dyes. Further, these dyes can also be combined with other anionic- or cationic-charged organic molecules. Suitable cationic dyes, which contain cationic charged organic molecules, include: 3-[(4-amino-6-bromo-5,8-dihydro-1-hydroxy-8-imino-5-oxo-2-naphtyl)amino]-N,N,N-trimethylanilinium chloride (CI 56059; Basic Blue No. 99-Trade name Arianor Steel Blue & Jaracol Steel Blue); mixtures of: 8-[(4-amino-3-nitrophenyl)azo]-7-hydroxy-N,N,N-trimethyl-2-naphthaleneaminium chloride [Major]; 8-[(4-amino-2-nitrophenyl)azo]-7-hydroxy-N,N,N-trimethyl-2-naphthaleneaminium chloride [Minor] (Basic Brown No. 17-Arianor Sienna Brown & Jaracol Sienna Brown); 8-[(4-aminophenyl)azo]-7-hydroxy-N,N, N-trimethyl-2-naphthaleneaminium chloride(CI 12250; Basic Brown No. 16-Arianor Mahogany & Jaracol Mahogany); 3-[4,5-dihydro-3-methyl-5-oxo-1-phenyl-1H-pyrazol-4-yl) azo]-N,N,N-trimethylanilinium chloride (CI 12719; Basic Yellow No. 57-Arianor Straw Yellow & Jaracol Straw Yellow); and 7-Hydroxy-8-[(2-methoxyphenyl)azo]-N,N,N-trimethyl-2-naphthaleneaminium chloride (CI 12245; Basic Red No. 76-Arianor Madder Red & Jaracol Madder Red). The dyes mentioned above are available from Warner Jenkinson Europe, Kings Lynn, Norfolk, UK. Jaracol dyes are available from James Robinson Dyes, Huddersfield, UK. Other examples of suitable cationic dyes containing cationic charged organic molecules suitable for use in the invention include: 9-(dimethylamino)benzo[a]phenoxazin-7-ium chloride (CI 51175; Basic Blue No. 6); di[4-(diethylamino) phenyl]-[4-(ethylamino)naphthyl]carbenium chloride (CI 42595; Basic Blue No. 7); 3,7-di-(dimethylaminophenothiazin-5-ium chloride (CI 52015; Basic Blue No. 9); di[4-(dimethylamino)phenyl-[4-(phenylamino)naphthyl]carbenium chloride (CI 44045; Basic Blue No. 26); 2-[(4-(ethyl(2-hydroxyethyl)amino)phenyl)azo]-6-methoxy-3-methylbenzothiazolium methyl sulfate (CI 11154; Basic Blue No. 41); bis[4-(dimethylamino)phenyl][4-(methylamino)phenyl]carbenium chloride (CI 42535; Basic Violet No. 1); tris-[4-(dimethylamino) phenyl]carbenium chloride (CI 42555; Basic Violet No. 3); 2-[3,6-(diethylamino)dibenzopyranium-9-yl]benzoic acid chloride (CI 45170; Basic Violet No. 10); di(4-aminophenyl)(4-amino-3-methylphenyl)carbenium chloride (CI 42510; Basic Violet No. 14); 1,3-bis[(2,4-diamino-5-methylphenyl) azo]-3-methylbenzene (CI-21010; Basic Brown No. 4); 1-[(4-amino-2-nitrophenyl)azo]-7-(trimethylammonio)-2-naphthol chloride (CI 12251; Basic Brown No. 17); 3,7-diamino-2,8-dimethyl-5-phenylphenazinium chloride (CI 50240; Basic Red No. 2); 1,4-dimethyl-5-[(4-(dimethylamino)phenyl)azo]-1,2,4-triazolium chloride (CI 11055; Basic Red No. 22); 2-[2-((2,4-dimethyoxyphenyl)amino)ethenyl]-1,3,3-trimethyl-3H-indol-1-ium chloride (CI 48055; Basic Yellow No. 11); and bis[4-(diethylamino)phenyl)phenylcarbenium hydrogen sulfate (1:1) (CI 42040; Basic Green No. 1). Charged organic molecules which are the cationic species in the so-called Basic dyes are particularly suitable. Suitable anionic dyes, which contain anionic charged organic molecules, include azo dyes, xanthene dyes and dyes based on carbenium salts. Specific examples of dyes are: Suitable anionic dyes, which contain anionic charged organic molecules, include azo dyes, xanthene dyes and dyes based on carbenium salts. Specific examples of dyes are: 6-hydroxy-5-[(4-sulfophenyl)azo]-2-naphthalenesulfonic acid disodium salt (CI 15985; Food Yellow No. 3); 2,4-dinitro-1-naphthol-7-sulfonic acid disodium salt (CI 10316; Acid Yellow No. 1; Food Yellow No. 1); 2-(2-quinolyl)-1H-indene-1,3(2H)-dione (mixture of mono- and disulfonic acid) (CI 47005; Food Yellow No. 13; Acid Yellow No. 3); 4,5-dihydro-5-oxo-1-(4-sulfophenyl)-4-[(4-sulfophenyl)azo]-1H-pyrazole-3-carboxylic acid trisodium salt (CI-19140; Food Yellow No. 4; Acid Yellow No. 23); 3′,6′-dihydroxyspiro[isobenzofuran-1(3H), 9-[9H]xanthen]-3-one disodium salt (CI 45350; Acid Yellow No. 73; D &C Yellow No. 8); 5-[(2,4-dinitrophenyl)amino]-2-phenylaminobenzenesulfonic acid sodium salt (CI-10385; Acid Orange No. 3); 4-[(2,4-dihydroxyphenyl)azo]benzenesulfonic acid monosodium salt (CI 14270; Acid Orange No. 6); 4-[(2-hydroxy-1-naphthalenyl)azo]benzenesulfonic acid monosodium salt (CI 15510; Acid Orange No. 7); 4-[[3-[(2,4-dimethylphenyl)azo]-2,4-dihydroxyphenyl]azo]benzenesulfonic acid monosodium salt (CI 20170; Acid Orange No. 24); 4-hydroxy-3-[(4-sulfo-1-naphthalenyl)azo]-1-naphthalenesulfonic acid disodium salt (CI 14720; Acid Red No. 14); 7-hydroxy-8-[(4-sulfo-1-naphthalenyl)azo]-1,3-naphthalenedisulfonic acid trisodium salt (CI 16255; Ponceau 4R; Acid Red No. 18); 3-hydroxy-4-[(4-sulfo-1-naphthalenyl)azo]-2,7-naphthalenedisulfonic acid trisodium salt (CI-16185; Acid Red No. 27; Food Red 9); 5-amino-4-hydroxy-3-(phenylazo)-2,7-naphthalenedisulfonic acid disodium salt (CI 17200; Acid Red No. 33); 5-(acetylamino)-4-hydroxy-3-[(2-methylphenyl)azo]-2,7-naphthalenedisulfonic acid disodium salt (CI 18065; Acid Red No. 35); 3′-6′-dihyroxy-2′,4′,5′,7′-tetraiodospiro[isobenzofuran-1(3H), 9′-[9H]xanthen]-3-one disodium salt (CI 45430; Acid Red No. 51); N-[6-(diethylamino)-9-(2,4-disulfophenyl)-3H-xanthen-3-ylidene]-N-ethylethaneaminium hydroxide, internal salt, sodium salt (CI 45100; Acid Red No. 52); 7-hydroxy-8-[[4-(phenylazo)phenyl]azo]-1,3-napthalenedisulfonic acid disodium salt (CI 27290; Acid Red No. 73); 2′,4′,5′,7′-tetrabromo-3′,6′-dihydroxyspiro[isobenzofurane-1(3H), 9′-[9H]-xanthen]-3-one disodium salt (CI 45380; Acid Red No. 87); 2′,4′,5′,7′-tetrabromo-4,5,6,7-tetrachloro-3′,6′,-dihydroxyspiro[isobenzofuran-1(3H), 9′-[9H]-xanthen]-3-one disodium salt (CI 45410; Acid Red No. 92); 3′,6′-dihydroxy-4′5′-diiodospiro[isobenzofuran]-1(3H), 9′-(9H)-xanthen]-3-one disodium salt (CI 45425; Acid Red No. 95); Benzenemethanaminium, N-ethyl-N-[4-[[4-[ethyl[(3-sulfophenyl)methyl]amino]phenyl](2-sulfophenyl)methylene]2,5-cyclohexadiene-1-ylidene]-3-sulpho-, hydroxide, inner salt disodium salt, CI-42090; Acid Blue No. 9); 2,2′-[(9,10-dihydro-9,19-dioxo-1,4-anthracenediyl)diimino]bis[5-methyl]benzenesulphonic acid disodium salt (CI 61570; Acid Green No. 25); N-[4-[[4-(diethylamino)phenyl](2-hydroxy-3,6-disulfo-1-napthalenyl)methylene]-2,5-cyclohexadien-1-ylidene]-N-methylmethaminium hydroxide internal salt, monosodium salt (CI 44090; Food Green No. 4; Acid Green No. 50); N-[4-[[4-(diethylamino)phenyl](2,4-disulfophenyl)methylene)-2,5-cyclohexadien-1-ylidene]-N-ethylethanaminium hydroxide internal salt, sodium salt (CI 42045; Food Blue No. 3; Acid Blue No. 1); N-[4-[[4-(diethylamino)phenyl](5-hydroxy-2,4-disulfophenyl)methylene]2,5-cyclohexadien-1-ylidene]-N-ethylethanaminium hydroxide internal salt, calcium salt (2:1)(CI 42051; Acid Blue No. 3); 1-amino-4-(cyclohexylamino)-9,10-dihydro-9,10-dioxo-2-anthracenesulfonic acid monosodium salt (CI 62045; Acid Blue No. 62); 2-(1,3-dihydro-3-oxo-5-sulfo-2H-indol-2-ylidene)-2,3-dihydro-3-oxo-1H-indole-5-sulfonic acid disodium salt (CI 73015, Acid Blue No. 74); 9-(2-carboxyphenyl)-3-[(2-methylphenyl)amino]-6-[(2-methyl-4-sulfophenyl)amino]xanthylium hydroxide internal salt, monosodium salt (CI 45190; Acid Violet No. 9); 2-[(9,10-dihydro-4-hydroxy-9,10-dioxo-1-anthracenyl)amino]-5-methylbenzenesulfonic acid monosodium salt (CI 60730; D &C Violet No. 2; Acid Violet No. 43); bis[3-nitro-4-[(4-phenylamino)-3-sulfophenylamino]phenyl]sulfone (CI 10410; Acid Brown No. 13); 4-amino-5-hydroxy-3-[(4-nitrophenyl)azo]-6-(phenylazo)-2,7-naphthalenedisulfonic acid disodium salt (CI 20470; Acid Black No. 1); 3-hydroxy-4-[(2-hydroxynaphth-1-yl) azo]-7-nitro-1-naphthalenesulfonic acid chromium complex (3:2) (CI 15711; Acid Black No. 52); 3-[(2,4-dimethyl-5-sulfophenyl) azo]-4-hydroxy-1-naphthalenesulfonic acid disodium salt (CI 14700; Food Red No. 1; Ponceau SX; FD &C Red No. 4); 4-(acetylamino)-5-hydroxy-6-[(7-sulfo-4-[(4-sulfophenyl]azo]-1-naphthalenyl)azo]-1,7-naphthalene disulfonic acid tetrasodium salt (CI-28440, Food Black No. 1); and 3-hydroxy-4-(3-methyl-5-oxo-1-phenyl-4,5-dihydro-1H-pyrazol-4-ylazo)naphthalene-1-sulfonic acid sodium salt, chromium complex (Acid Red No. 195). Charged anionic organic molecules, which are the anionic species in the so-called Acid dyes are particularly preferred. The charged organic molecule is preferably selected from the anion of anionic surfactant, an anionic polymer and a polyelectrolyte, when a cationic clay is used. The preferred anionic charged organic molecule is the anion of an anionic surfactant. Examples of suitable anionic surfactants are the alkyl sulphates, alkyl ether sulphates, alkaryl sulphonates, alkanoyl isethionates, alkyl succinates, alkyl sulphosuccinates, alkyl phosphates, alkyl ether phosphates, alkyl carboxylates, alkyl ether carboxylates, alkyl ester carboxylates, N-alkyl sarcosinates, and alpha-olefin sulphonates, especially their sodium, magnesium, ammonium and mono-, di- and triethanolamine salts. The alkyl and acyl groups generally contain from 8 to 22, preferably 12 to 22 carbon atoms, and may be saturated or unsaturated and can contain, in addition to carbon and hydrogen atoms, ether linkages, and other groups such as amino and ester groups. The alkyl ether sulphates, alkyl ether phosphates and alkyl ether carboxylates may contain from 1 to 10 ethylene oxide or propylene oxide units per molecule. Typical anionic surfactants for use in compositions of the invention include sodium oleyl sulpho succinate, ammonium lauryl sulphosuccinate, ammonium lauryl sulphate, sodium cocoyl isethionate, sodium lauryl isethionate and sodium N-lauryl sarcosinate, sodium lauryl sulphate, sodium lauryl ether sulphate(n)EO, (where n ranges from 1 to 3), ammonium lauryl sulphate, ammonium lauryl ether sulphate(n)EO, (where n ranges from 1 to 3), sodium heptadecyl sulphate, sodium and tetra decyl sulphate. Further suitable anionic charged organic molecules are anionic polymers. Examples of suitable anionic polymers are polyacrylates, cross-linked polyacrylates, hydrophobically modified polyacrylates, polyalkylacrylates, polymethacrylates, polymethylvinylether/maleic anhydride (PVM/MA) copolymers, alkyl esters of PVM/MA copolymers, monoester resins of PVM/MA copolymers, polymethylcarboxylates, polysulphonates and polyphosphates.

The silicone vesicle can be combined with known surfactants to form emulsified formulation or provide cleansing effect of the formulation. Known surfactants include anionic, cationic, nonionic and amphoteric surfactants, but are not particularly limited to these. Any of those which are commonly used in cosmetics may be used. Specific examples are as follows: anionic surfactants including fatty acid soaps, such as sodium stearate and triethanolamine palmitate, alkylether carboxylic acids and salts thereof, carboxylates of condensates from amino acids and fatty acids, alkyl sulfonic acids, alkenesulfonates, fatty acid ester sulfonates, fatty acid amide sulfonates, sulfonate salts of the formalin condensates with alkyl sulfonates, salts of sulfate esters such as salts of alkyl sulfates, salts of secondary higher alcohol sulfates, salts of alkyl/allyl ether sulfates, salts of fatty acid ester sulfates, salts of fatty acid alkylolamide sulfates, and Turkey Red oil, alkyl phosphates, ether phosphates, alkylallylether phosphates, amide phosphates, and N-acylamino surfactants; cationic surfactants including amine salts such as alkylamine salts, polyamine and amino alcohol fatty acid derivatives, alkyl quaternary ammonium salts, aromatic quaternary ammonium salts, pyridium salts and imidazolium salts; nonionic surfactants including sorbitan fatty acid esters, glycerin fatty acid esters, polyglycerin fatty acid esters, propylene glycol fatty acid esters, polyethylene glycol fatty acid esters, sucrose fatty acid esters, polyoxyethylene alkylethers, polyoxypropylene alkylethers, polyoxyethylene alkylphenylether, polyoxyethylene fatty acid esters, polyoxyethylene sorbitan fatty acid esters, polyoxyethylene sorbitol fatty acid esters, polyoxyethylene glycerin fatty acid esters, polyoxyethylene propylene glycol fatty acid esters, polyoxyethylene castor oil, polyoxyethylene hydrogenated castor oil, polyoxyethylene phytostanolether, polyoxyethylene phytosterolether, polyoxyethylene cholestanolether, polyoxyethylene cholesterylether, alkanolamide, sugar ethers, and sugar amides; and amphoteric surfactants including betaine, aminocarboxylates, and imidazoline derivatives.

A desirable amount of the surfactant to be added ranges from 0.1 to 20 mass %, preferably from 0.5 to 10 mass % relative to the total amount of the total formulation. One or more kinds of the surfactants may be used.

The silicone vesicle compositions can be combined with germicides to keep its bactericidal effect on skin or hair. The combination of the silicone vesicle with germicides give good retention of its effect, waterproofing and sweat resistance. Further, adding these germicides as deodorant with the silicone compositions provide a keep-well and retentive formulation. Suitable germicides include benzalkonium chloride, benzethonium chlorohexidine chloride, cetyl pyridinium chloride, chlorhexidine gluconate, chlorhexidine acetate, chlorhexidine hydrochloride, triclosan, triclocarban, isopropylmethylphenol, hinokitiol (B-Thujaplicin), zinc pyrithione, piroctone olamine, resorcin, phenol, sorbic acid, hexachlorophenone, salicylic acid, silver-carrying zeolite, or silver-carrying silica. Preferably, silver-carrying zeolite and silver-carrying silica may be used. One or more kinds of the germicides may be used. For the antiseptics, alkyl paraoxybenzoates, benzoic acid, sodium benzoate, sorbic acid, potassium sorbate, and phenoxyethanol may be used. For the antibacterial agents, benzoic acid, salicylic acid, carbolic acid, sorbic acid, paraoxybenzoic acid alkyl esters, parachloromethacresol, hexachlorophene, benzalkonium chloride, chlorohexydine chloride, trichlorocarbanilide, triclosan, photosensitizer and phenoxyethanol may be used.

### EXAMPLES

These examples are intended to illustrate the invention to one of ordinary skill in the art and should not be interpreted as limiting the scope of the invention set forth in the claims.

### Materials

The following silicone polyethers were used in the representative examples. The silicone polyethers were prepared by well known platinum catalyzed hydrosilylation reaction techniques.
SPE 1=rake type SPE having an average formula of MD₉₄D^{(EO12)}₆M, prepared from MD₉₄D^{H}₆M siloxane and AE501 monoallyloxy polyether with 12 EO units.
SPE 2=(AB)n block copolymer prepared from the hydrosilylation reaction of M^{H}D₅₀ M^{H} siloxane and Polyglycol AA1200 polyether.
SPE 3=a rake SPE having an average formula of MD₇₀D^{(EO12)}₃M.

### Example 1

### Rake SPE Vesicles with Vitamin C Dispersion

A solution of ascorbic acid (vitamin C) in propylene glycol was prepared first, and then incorporated into SPE 1 to form a homogeneous dispersion. Ethanol (EtOH) was added into the mixture to form a homogeneous mixture. Water was then gradually incorporated into the SPE 1/vitamin C/propylene glycol/EtOH mixture. The rate of water introduction was controlled such that the mixture remained homogeneous at all time. The SPE molecules arranged themselves into vesicles, and the vitamin C active was encapsulated within the interior of the vesicles.

TABLE 1

| Example # | |
|---|---|
| 1A | 1B |

| | | |
|---|---|---|
| Process History | Mixed | Mixed, Rotovap stripped |
| SPE 1 | 30.32 | 28.87 |
| Vitamin C (ascorbic acid) | 6.031 | 5.74 |
| Propylene Glycol | 6.272 | 5.97 |
| EtOH | 90.54 | 86.21 |
| Water | 181.1 | 172.44 |
| Volatiles Removed, g | 0 | 77.6 |
| Batch size, g | 283.947 | 221.63 |
| Wt. % SPE | 9.65 | 13.03 |
| Wt. % Vitamin C | 1.92 | 2.59 |
| Wt. % Propylene Glycol | 2.00 | 2.69 |
| Wt. % EtOH | 28.81 | 3.89 |
| Wt. % Water | 57.63 | 77.80 |
| Wt % Loading | 16.59 | 16.59 |
| Appearance | Slightly hazy | Hazy, very fluid |
| mv Avg. size, μm | 0.077 | 0.082 |
| D (v, 0.5), μm | 0.074 | 0.078 |
| D (v, 0.9), μm | 0.108 | 0.114 |

In this example, hydrophilic vitamin C (ascorbic acid) active was introduced in the form of a solution in propylene glycol into SPE 1, and ethanol solvent was introduced to yield a homogeneous dispersion of SPE/vitamin C/propylene glycol in EtOH. Water was subsequently introduced to form vesicle dispersion in EtOH/water. The EtOH was mostly removed by vacuum stripping using a Rotovap stripper. The final vesicle and vitamin C composition in the dispersion is summarized in Table 1. The final dispersion was a hazy, fluid mixture with an average vesicle size of about 82 nm.

### Example 2

### Rake SPE Vesicles with Vitamin C Dispersion

Vitamin C encapsulated silicone vesicles in ethanol solvent free dispersion was also prepared. This example is intended to demonstrate that the volatile ethanol solvent used in this invention serves primarily as a processing aid and is not required in the final vesicle dispersion.

Vitamin C was incorporated in propylene glycol solution. The rake SPE, vitamin C/propylene glycol solution and ethanol solvent were mixed to form homogeneous mixture. Water was then introduced while the mixture was under continuous stirring to maintain a homogeneous state. Silicone vesicles were formed during the water addition and some portion of vitamin C was encapsulated at inside of the vesicles.

Ethanol was removed using a Rotovap stripper under vacuum at room temperature. The final vesicle composition and the amount of vitamin C in the vesicle dispersion is shown in Table 2.

TABLE 2

| | Example # | |
|---|---|---|
| | 2A | 2B |
| Mixing method | All ingredients except water | Sample stripped to |
| | all added at the beginning | 0% EtOH |
| Process History | Mixed | Mixed, Rotovap |
| | | stripped |
| SPE 1 g | 30.08 | 7.76 |
| Vitamin C (ascorbic | 6.005 | 1.55 |
| acid) | | |
| Propylene Glycol, g | 6.047 | 1.56 |
| EtOH, g | 90.235 | 23.29 |
| Water, g | 180.412 | 46.57 |
| Volatiles Removed, g | 0 | 30.95 |
| Batch size, g | 312.78 | 49.78 |
| Wt. % SPE Polymer | 9.62 | 15.59 |
| Wt. % Vitamin C | 1.92 | 3.11 |
| Wt. % Propylene | 1.93 | 3.13 |
| Glycol | | |
| Wt. % EtOH | 28.85 | 0.00 |
| Wt. % Water | 57.68 | 78.16 |
| wt % Loading | 16.64 | 16.63 |
| mv Avg. size, μm | 0.060 | 0.067 |
| D (v, 0.5), μm | 0.057 | 0.065 |
| D (v, 0.9), μm | 0.094 | 0.093 |

The vitamin C encapsulated, ethanol-free silicone vesicles exhibited excellent stability. After 8 months of standing at room temperature, the 2B vesicle dispersion remained homogeneous with light amber milky appearance.

### Example 3

### Rake SPE Vesicles with Vitamin C and Vitamin A Palmitate Dispersion

A hydrophobic active (vitamin A palmitate, or VAP) and hydrophilic active (vitamin C) were encapsulated in silicone vesicles in water-continuous dispersion, following the method detailed in this invention. A mixture of VAP/BHT (butylated hydroxytoluene) stabilizer/Dow Corning 245 fluid (D5) fluid @50/1.5/48.5 by weight was first prepared. Vitamin C solid was dissolved in 1,2-propanediol to form a clear solution. The VAP and vitamin C solutions were then incorporated into the selected rake SPE 1 and ethanol solvent were all mixed together to give a homogeneous mixture. Water was then introduced while under mixing to form silicone vesicles both actives encapsulated at the same time. The vesicle dispersion was then passed through a MicroFluidizer (or equivalent high-shear device like Homogenizer) to further reduce the vesicle size and particle size dispersity.

As the last step, volatile ethanol solvent was removed via a Rotovap stripper under vacuum at room temperature. The final compositions of the SPE vesicle, VAP and vitamin C actives in the dispersion are summarized in Table 3.

TABLE 3

| | Example 3 | | |
|---|---|---|---|
| | 3A | 3B | 3C |
| Mixing method | All ingredients except | Same | Same |
| | water added at the | | |
| | beginning | | |
| Process History | Mixed | Mixed, | Mixed, |
| | | MicroFluidized | MicroFluidized; |
| | | | Rotovap stripped |
| SPE 1, g | | 30.169 | 27.046 |
| VAP, g | 5.070 | 5.070 | 4.558 |
| DC 245 Fluid, g | 4.922 | 4.922 | 4.406 |
| BHT, g | 0.152 | 0.152 | 0.152 |
| Vitamin C | | 6.224 | 5.592 |
| (ascorbic acid) | | | |
| 1,2-Propanediol, g | 6.272 | 6.272 | 5.622 |
| EtOH, g | 90.190 | 90.190 | 80.896 |
| Water, g | 185.700 | 185.700 | 166.532 |
| Volatiles Removed, g | 0 | 0 | 79.20 |
| Batch size, g | 328.699 | 328.699 | 224.69 |
| Wt. % Polymer | 8.90 | 8.90 | 12.54 |
| Wt. % VAP | 1.50 | 1.50 | 2.11 |
| Wt. % Vitamin C | 1.84 | 1.84 | 2.59 |
| Wt. % BHT | 0.05 | 0.05 | 0.07 |
| Wt. % DC 245 Fluid | 1.45 | 1.45 | 2.04 |
| Wt. % 1,2-Propanediol | 1.85 | 1.85 | 2.61 |
| Wt. % EtOH | 26.62 | 26.62 | 0.79 |
| Wt. % Water | 54.80 | 54.80 | 77.24 |
| Vit. C wt % Loading | 17.13 | 17.13 | 17.12 |
| VAP wt % Loading | 14.42 | 14.42 | 14.40 |
| Appearance | | | Very fluid; very pale |
| | | | yellow, almost white |
| mv Avg. size, μm | 3.380 | 0.057 | 0.064 |
| D (v, 0.5), μm | 5.610 | 0.042 | 0.055 |
| D (v, 0.9), μm | 6.290 | 0.094 | 0.109 |

The vitamin A palmitate (VAP) and vitamin C (ascorbic acid) encapsulated silicone vesicles is a pale yellowish milky dispersion and has a very fluid consistency. The VAP and vitamin C encapsulated vesicles have an average size of 64 nm in diameter.

### Examples 4 and 5

### (AB)n SPE Block Copolymer Vesicles with Vitamin C Dispersions

The method of the present invention was also used for simultaneous encapsulation of hydrophilic actives and other polar substances into silicone vesicles formed from a (AB)n SPE block copolymer (SPE 2). The process and resulting formulations are summarized in Tables 4 and 5

TABLE 4

| | Example # | | |
|---|---|---|---|
| | 4A | 4B | 4C |
| Mixing method | All ingredients | Same | Same |
| | except water | | |
| | added at | | |
| | the beginning | | |
| Process History | Mixed | Mixed, | Mixed, |
| | | MicroFluidized | MicroFluidized, |
| | | | and Rotovap |
| | | | stripped |
| SPE 2, g | 31.21 | 31.21 | 26.93 |
| Vitamin C | 6.342 | 6.342 | 5.47 |
| (ascorbic acid) | | | |
| Propylene Glycol, g | 6.157 | 6.157 | 5.31 |
| EtOH, g | 90.940 | 90.940 | 78.46 |
| Water, g | 181.0 | 181.0 | 156.15 |
| Volatiles | 0 | 0 | 73.8 |
| Removed, g | | | |
| Batch size, g | 315.65 | 315.65 | 198.52 |
| Wt. % SPE | 9.89 | 9.89 | 13.56 |
| Wt. % Vitamin C | 2.01 | 2.01 | 2.76 |
| Wt. % Propylene | 1.95 | 1.95 | 2.68 |
| Glycol | | | |
| Wt. % EtOH | 28.81 | 28.81 | 2.35 |
| Wt. % Water | 57.34 | 57.34 | 78.66 |
| Wt % Loading | 16.89 | 16.89 | 16.89 |
| mv Avg. size, μm | 0.628 | 0.077 | 0.090 |
| D (v, 0.5), μm | 0.191 | 0.057 | 0.075 |
| D (v, 0.9), μm | 2.025 | 0.150 | 0.150 |
| Appearance after 6 | | | Homogeneous |
| months | | | dispersion with |
| | | | light amber |
| | | | appearance |
| Wt. % Vitamin C | | | 1.80 |
| after 2 months, | | | |
| per HPLC | | | |

The vitamin C encapsulated silicone vesicles derived from the (AB)n SPE 2 block copolymer exhibited excellent stability. The vitamin C as measured by HPLC after 2 months of aging was 1.80% by weight, corresponding to about 65% of the theoretical amount.

TABLE 5

| | Example # | | |
|---|---|---|---|
| | 5A | 5B | 5C |
| Mixing method | All ingredients except | Same | Same |
| | water added at the | | |
| | beginning | | |
| Process History | Mixed | Mixed, | Mixed, |
| | | MicroFluidized | MicroFluidized, and |
| | | | Rotovap stripped |
| SPE 2 g | 30.84 | 30.84 | 27.91 |
| Vitamin C (ascorbic acid) | 6.389 | 6.389 | 5.78 |
| Propylene glycol, g | 6.382 | 6.382 | 5.78 |
| EtOH, g | 90.953 | 90.953 | 82.31 |
| Water, g | 181.1 | 181.1 | 163.88 |
| Volatiles Removed, g | 0 | 0 | 84.4 |
| Batch size, g | 315.64 | 315.64 | 201.26 |
| Wt. % SPE | 9.77 | 9.77 | 13.87 |
| Wt. % Vitamin C | 2.02 | 2.02 | 2.87 |
| Wt. % Propylene glycol | 2.02 | 2.02 | 2.87 |
| Wt. % EtOH | 28.82 | 28.82 | 0.00 |
| Wt. % Water | 57.37 | 57.37 | 80.39 |
| Wt. % Loading | 17.16 | 17.16 | 17.16 |
| Appearance | | | Homogeneous milky |
| | | | dispersion |
| Mv Avg. size, μm | 0.176 | 0.097 | 0.105 |
| D (v, 0.5), μm | 0.142 | 0.078 | 0.079 |
| D (v, 0.9), μm | 0.298 | 0.159 | 0.171 |
| Appearance after 6 months | | | Homogeneous |
| at room temperature | | | dispersion with |
| | | | beige appearance |

The vitamin C encapsulated silicone vesicles prepared in this example is a homogeneous milky dispersion with an average diameter of 0.105 μm. The vesicle dispersion remained homogeneous with slight discoloration after 6 months.

### Example 6

### (AB)n SPE Block Copolymer Vesicles with Vitamin C and Vitamin A Dispersions

Vitamin A palmitate (lipophilic) and vitamin C (hydrophilic) actives were both encapsulated into silicone vesicles derived from (AB)ₙ silicone polyether block copolymer. VAP was first made in a mixture of BHT and 245 fluid, and vitamin C was dissolved in propylene glycol. Both were then incorporated with the (AB)ₙ SPE copolymer and ethanol to form a homogeneous solution. The composition of the final dispersion is summarized Table 6.

TABLE 6

| | Vitamin A | Example # | | |
|---|---|---|---|---|
| | Premix | 6A | 6B | 6C |
| Mixing method | | All ingredients | Same | Same |
| | | except EtOH and | | |
| | | water added at the | | |
| | | beginning | | |
| Process History | Mixed | Mixed | Mixed, | Mixed, |
| | | | MicroFluidized | MicroFluidized; |
| | | | | Rotovap stripped |
| SPE 2, g | | 32.820 | 32.820 | 28.360 |
| Vitamin A Palmitate | | 11.100 | 11.100 | |
| Premix, g | | | | |
| VAP, g | 6.09 | | | 4.653 |
| DC 245 Fluid, g | 6.151 | | | 4.699 |
| BHT, g | 0.314 | | | 0.240 |
| Vitamin C (ascorbic acid) | | 6.230 | 6.230 | 5.383 |
| Propylene Glycol, g | | 6.080 | 6.080 | 5.254 |
| EtOH, g | | 90.540 | 90.540 | 78.236 |
| Water, g | | 181.270 | 181.270 | 156.636 |
| Volatiles Removed, g | | 0 | 0 | 71.70 |
| Batch size, g | | 284.120 | 284.120 | 211.76 |
| Wt. % SPE polymer | 0.00 | 10.00 | 10.00 | 13.39 |
| Wt. % VAP | 48.51 | 1.64 | 1.64 | 2.20 |
| Wt. % Vitamin C | 0.00 | 1.90 | 1.90 | 2.54 |
| Wt. % BHT | 2.50 | 0.08 | 0.08 | 0.11 |
| Wt. % 245 Fluid | 48.99 | 1.66 | 1.66 | 2.22 |
| Wt. % Propylene Glycol | | 1.85 | 1.85 | 2.48 |
| Wt. % EtOH | 0.00 | 27.60 | 27.60 | 3.09 |
| Wt. % Water | 0.00 | 55.26 | 55.26 | 73.97 |
| Vit. C wt % Loading | | 15.95 | 15.95 | 15.95 |
| VAP wt % Loading | | 14.09 | 14.09 | 14.09 |
| mv Avg. size, μm | | 1.467 | 0.089 | 0.216 |
| D (v, 0.5), μm | | 1.587 | 0.080 | 0.095 |
| D (v, 0.9), μm | | 2.125 | 0.147 | 0.692 |
| Wt. % VAP after | | | | 1.90 |
| 2 months, per HPLC | | | | |
| Wt. % Vitamin C after | | | | 1.40 |
| 2 moths, per HPLC | | | | |

The VAP and vitamin C actives encapsulated silicone vesicles had an average size of 0.216 μm in water, The amount of VAP and vitamin C actives detected, per HPLC assays, were 1.90 and 1.40% by weight, respectively. They are 86% and 55% of the theoretical inputs respectively.

### Example 7

### Rake SPE Vesicles with Vitamin C Dispersion from Highly Hydrophobic SPE with Homogenization Process

The method of making vitamin C encapsulated silicone vesicles from a highly hydrophobic silicone polyether is shown. This SPE has very poor to negligible solubility in water. Vitamin C in the form of propylene glycol solution was incorporated into silicone vesicles following the method described above. The final composition of silicone vesicles prepared by mixing and then Rotovap vacuum strip was demonstrated. No MicroFluidizer or a high shear homogenizer was used in making this vesicle.

TABLE 7

| | Example # | | |
|---|---|---|---|
| | 7A | 7B | 7C |
| Process History | Mixed | Mixed, | Mixed, |
| | | MicroFluidized | MicroFluidized, |
| | | | Rotovap stripped |
| SPE 3 g | 30.07 | 30.07 | 11.76 |
| Vitamin C | 6.255 | 6.255 | 2.45 |
| (ascorbic acid) | | | |
| Propylene Glycol | 6.051 | 6.051 | 2.37 |
| EtOH | 90.41 | 90.41 | 35.37 |
| Water | 180.714 | 180.714 | 70.71 |
| Volatiles Removed, g | 0 | 0 | 33.36 |
| Batch size, g | 313.49 | 313.49 | 89.3 |
| Wt. % SPE Polymer | 9.59 | 9.59 | 13.17 |
| Wt. % Vitamin C | 2.00 | 2.00 | 2.74 |
| Wt. % Propylene | 1.93 | 1.93 | 2.65 |
| Glycol | | | |
| Wt. % EtOH | 28.84 | 28.84 | 2.25 |
| Wt. % Water | 57.65 | 57.65 | 79.18 |
| Wt % Loading | 17.22 | 17.22 | 17.22 |
| Appearance | Slightly | Very hazy with | Cloudy with an off- |
| | hazy fluid | a yellow tint | white to yellow tint |
| pH | 3.17 | 3.19 | 2.69 |
| mv Avg. size, μm | 0.094 | 0.033 | 0.039 |
| D (v, 0.5), μm | 0.085 | 0.030 | 0.037 |
| D (v, 0.9), μm | 0.149 | 0.051 | 0.059 |

### Example 8

### Rake SPE Vesicles with Vitamin C Dispersion from Highly Hydrophobic SPE without Homogenization Process

Vitamin C encapsulated silicone vesicles were made by dispersing the corresponding components, as summarized in Table 8. The hydrophilic active was encapsulated within the silicone vesicles as they formed during the dispersion stage. Rotovap vacuum stripper was used to remove the volatile ethanol solvent. No MicroFluidizer or a high shear homogenizer was used in making this vesicle.

TABLE 8

| | Example # | |
|---|---|---|
| | 8A | 8B |
| Mix Method | All ingredients | Same |
| | except water | |
| | all added at | |
| | the beginning | |
| Process History | Mixed | Mixed, Rotovap stripped |
| SH3775M | 30.07 | 11.90 |
| Vitamin C | 6.255 | 2.48 |
| (ascorbic acid) | | |
| Propylene Glycol | 6.051 | 2.40 |
| EtOH | 90.41 | 35.79 |
| Water | 180.714 | 71.55 |
| Volatiles Removed, g | 0 | 36.16 |
| Batch size, g | 313.49 | 87.96 |
| Wt. % Polymer | 9.59 | 13.53 |
| Wt. % Vitamin C | 2.00 | 2.82 |
| Wt. % Propylene Glycol | 1.93 | 2.72 |
| Wt. % EtOH | 28.84 | 0.00 |
| Wt. % Water | 57.65 | 80.92 |
| Wt % Loading | 17.22 | 17.22 |
| Appearance | Slightly hazy fluid | Hazy fluid |
| pH | 3.17 | 2.84 |
| Mv Avg. size, μm | 0.094 | 0.095 |
| D (v, 0.5), μm | 0.085 | 0.089 |
| D (v, 0.9), μm | 0.149 | 0.142 |
| Appearance after 9 | | Homogeneous dispersion |
| months @ RT | | with beige appearance |

The vitamin C encapsulated silicone vesicles derived from SPE 3 of this example had an average size of about 95 nm. This vitamin C encapsulated silicone vesicles remained homogeneous with beige appearance after 9 months of standing at room temperature.

### Example 9

### (Reference) Preparation of Vesicle Compositions

A vesicle composition (labeled as 9A) was prepared from a rake silicone polyether, having a nominal structure of MD₉₄D^{(EO12)}₆M, where M represents (CH₃)₃SiO_{1/2} siloxy units, D represent (CH₃)₂SiO siloxy units, (CH₃)R^{EO12}SiO siloxy units where REO¹² represents the polyethylene oxide group having the average formula, —CH₂CH₂CH₂—O—(CH₂CH₂O)₁₂H. A vesicle composition (labeled as 9B) was also prepared from a (AB)ₙ SPE block copolymer of M′D₅₀M′ siloxane (where M represents (CH₃)₂HSiO_{1/2} siloxy units, D represent (CH₃)₂SiO siloxy units) and Polyglycol AA1200 polyether (α,ω-diallyl polyethylene oxide having an average molecular weight (M_{w}) of 1200). Both vesicle compositions were processed to entrap vitamin A palmitate as a representative example of an active material. These vesicle compositions were made via processing in ethanol/water media. The composition and initial vesicle properties are summarized in Table 9. These vesicle compositions were used to prepare the personal care formulations of Examples 10-15.

TABLE 9

| | Vesicle Example ID | |
|---|---|---|
| | 9A | 9B |
| SPE type | rake SPE | (AB)ₙ SPE |
| Wt. % silicone polyether | 20.90 | 21.27 |
| Wt. % vitamin A palmitate | 4.50 | 4.59 |
| Wt. % carrier fluid + additive | 1.81 | 1.84 |
| Wt. % Water | 72.80 | 72.30 |
| Wt % Loading | 17.71 | 17.77 |
| Initial property | | |
| Vesicle dispersion appearance | Pale yellow fluid | Very pale yellow fluid |
| Average size Mv, μm | 0.0735 | 0.255 |
| D (v, 0.5), μm | 0.0592 | 0.2288 |
| D (v, 0.9), μm | 0.1361 | 0.447 |

### Example 10

### Moisturizing Gel

Part A

| | | |
|---|---|---|
| 1. | Water | q.s. |
| 2. | DMDM Hydantoin (Nipaguard DMDMH, Clariant GmbH) | 0.30% |
| 3. | Acrylates/C10-30 Alkyl acrylate cross polymer | 1% |
| 4. | (Carbopol ETD 2020, Noveon) | |
| 5. | Triethanolamine (30%) | q.s. |

Part B

| | | |
|---|---|---|
| 6. | Silicone Vesicles of Example 9 | q.s. |

### Example 11

Moisturizing gel

Part A

| | | |
|---|---|---|
| 1. | Water | q.s. |
| 2. | DMDM Hydantoin (Nipaguard DMDMH, Clariant GmbH) | 0.30% |
| 3. | Polyacrylamide, C13-14 Isoparafin, Laureth-7 | 1% |
| | (Sepigel 305, Seppic) | |

Part B

| | | |
|---|---|---|
| 4. | Silicone Vesicles of Example 9 | q.s. |

### Procedure:

Prepare gel in Part A

Add part B

Mix to homogeneous

### Example 12

### O/W Body Lotion

An oil-in-water body lotion is prepared following the following procedure. Silicone vesicles can also be formulated into other oil-in-water type cosmetic formulations.

Part A

| | | |
|---|---|---|
| 1. | Cetearyl Alcohol (Lanette O, Cognis) | 3% |
| 2. | Diisopropyl Adipate (Crodamol DA, Croda) | 5% |
| 3. | Dimethicone (Dow Corning ® 200 fluid, 100 cSt) | 0.5% |
| 4. | Potassium cetyl phosphate (Amphisol K, Roche | 1.5% |
| | Vitamins) | |
| 5. | Buthylated hydroxytoluene (BHT) | 0.05% |
| 6. | Tetrasodium EDTA | 0.1% |
| 7. | Phenoxyethanol, methyl paraben, ethyl paraben, propyl | 0.6% |
| | paraben, butylparaben (Phenonip, Clariant) | |

Part B

| | | |
|---|---|---|
| 8. | Water | q.s. |
| 9. | Carbomer (1%) (Carbopol 980, Noveon) | 30% |
| 10. | Potassium hydroxyde (10%) | 1.5% |

Part C

| | | |
|---|---|---|
| 11. | Silicone Vesicles of Example 9 | q.s. |

### Procedure

Heat Part A to 85° C. while stirring

When homogeneous, add Part B at 40° C.

Let cool down to room temperature and compensate for water loss

Add part C to AB

Mix to homogeneous

### Example 13

### W/O Radiant Beauty Formulation

Silicone vesicles are formulated into water-in-oil radiant beauty formulation following the procedures listed below. Other water-in-oil type personal care formulations can also be prepared following these similar steps.

Part A

| | | |
|---|---|---|
| 1. | Ethylhexyl Methoxycinnamate (Parsol MCX, Roche | 3% |
| | Vitamins) | |
| 2. | Butyl Methoxydibenzoylmethane (Parsol 1789, Roche | 1.5% |
| | Vitamins) | |
| 3. | Glyceryl Stearate, PEG 100 stearate (Arlacel 165, | 4% |
| | Uniqema) | |
| 4. | Butyrospermum Parkii, Shea butter (Cetiol SB 45, | 1% |
| | Cognis) | |
| 5. | Stearyl dimethicone (Dow Corning ® 2503 Cosmetic | 3% |
| | wax) | |
| 6. | Cetyl alcohol | 1% |
| 7. | Simmondsia Chinensis (Jojoba) Seed Oil | 4% |
| 8. | Lanolin oil (Fluilan, Croda) | 3% |
| 9. | Cyclomethicone (Dow Corning ® 245 Fluid) | 8% |
| 10. | Phenoxyethanol, methyl paraben, ethyl paraben, | 0.5% |
| | propyl paraben, butylparaben (Phenonip, Clariant) | |
| 11. | Cyclomethicone (and) Dimethicone Crosspolymer | 5% |
| | (Dow Corning ® 9045 Silicone Elastomer Blend) | |

Part B

| | | |
|---|---|---|
| 12. | Glycerin | 2% |
| 13. | Water | q.s. |

Part C

| | | |
|---|---|---|
| 14. | Polyacrylamide, C13-14 Isoparafin, Laureth-7 | 4% |
| | (Sepigel 305, Seppic) | |

Part D

| | | |
|---|---|---|
| 15. | Silicone Vesicles of Example 9 | q.s. |

### Procedure

Melt ingredients 1 and 2 at 60° C.
Add ingredients 3, 4, 5, 6 in order at 60° C., ensuring that each ingredient is melted before incorporating the next

### Add 7, 8, 9 and 10

Add ingredient 11 to form Part A

### Add Part B to Part A at 1500 rpm

Let cool down to room temperature
Add Part C to AB in “one shot” while stirring at maximum speed
Cease agitation immediately once viscosity increases

### Add Part D to ABC

Mix to homogeneous

### Example 14

### Mild Foundation

Color cosmetic products are prepared from silicone vesicles and other cosmetic pigments and colorants. Illustrated in this example is a mild foundation containing silicone vesicles, following the procedure shown bellow.

Part A

| | | |
|---|---|---|
| 1. | Polyglyceryl-4 Caprate (and) Sucrose Stearate (and) Sucrose Distearate (and) PEG-8 | 5.00% |
| | (and) Ammonium Polyacrylate (and) Mica (and) Tocopheryl Acetate (and) Macadamia | |
| | Ternifolia Seed Extract (Covacream, Sensient Cosmetic Technology - LCW) | |

Part B

| | | |
|---|---|---|
| 2. | Hydrogenated Polyisobutene (Squatol S, Sensient Cosmetic Technology - LCW) | 10.00% |
| 3. | Cyclomethicone (Dow Corning ® 245 Fluid) | 5.00% |

Part C

| | | |
|---|---|---|
| 4. | Glycerin | 2.50% |
| 5. | DMDM Hydantoin (Nipaguard DMDMH, Clariant GmbH) | 0.30% |
| 6. | Sodium polyacrylate (Covacryl J22, Sensient Cosmetic Technology - LCW) | 0.30% |
| 7. | Water | q.s. |

Part D

| | | |
|---|---|---|
| 8. | Red iron oxide (AQ 70401, Sensient Cosmetic Technology - LCW) | 0.50% |
| 9. | Yellow iron oxide (AQ 70402, Sensient Cosmetic Technology - LCW) | 0.90% |
| 10. | Black iron oxide (AQ 70403, Sensient Cosmetic Technology - LCW) | 0.10% |
| 11. | Titanium Dioxide (AQ 70409, Sensient Cosmetic Technology - LCW) | 8.50% |

Part E

| | | |
|---|---|---|
| 12. | Silicone Vesicles of Example 9 | q.s. |

### Procedure

Mix ingredients of Part B together

Add Part B to Part A under stirring—Part AB will look heterogeneous

Mix Part C ingredients together

Add Part C to Part AB and stir until homogeneous

Mix Part D ingredients together

Add Part D to the batch, stir under high shear during 30 minutes

When this is done, add Part E

Mix to homogeneous

### Example 15

### Emulsion Lipstick

This example illustrates how silicone vesicles can be used in formulating color cosmetic products including lipstick.

Part A

| | | |
|---|---|---|
| 1. | Lauryl PEG/PPG-18/18 Methicone (Dow Corning 5200 Formulation Aid) | 3.70% |
| 2. | Phenyl Trimethicone (Dow Corning 556 Cosmetic Grade Fluid) | 1.00% |
| 3. | Hexyl Laurate (Cetiol A, Cognis Corporation, Care Chemicals) | 3.50% |
| 4. | Disteardimonium Hectorite (Bentone 38, Elementis Specialties) | 0.30% |
| 5. | Isononyl Isononanoate (and) Polybutene (and) Pentaerythrityl Tetraisostearate (and) | 2.40% |
| | Isostearyl Alcohol (Covaclear, Sensient Cosmetic Technology - LCW) | |
| 6. | Iron Oxides (Unipure Red LC 381 AS-EM, Sensient Cosmetic Technology - LCW) | 3.00% |

Part B

| | | |
|---|---|---|
| 7. | Pure water | q.s. |
| 8. | Algae Extract (and) Sorbitol (Fucosorb, Sensient Cosmetic Technology - LCW) | 0.60% |
| 9. | Propylene glycol | 1.20% |

Part C

| | | |
|---|---|---|
| 10. | Ozokerite (and) Copernicia Cerifera (Carnauba) Wax (and) Euphorbia Cerifera | 20.00% |
| | (Candelilla) Wax (and) Paraffin (and) Butyl Stearate (and) Isopropyl Palmitate | |
| | (and) Mineral Oil (and) Ethylene/VA Copolymer (Covalip 94, Sensient Cosmetic | |
| | Technology - LCW) | |
| 11. | Ethylhexyl Hydroxystearate (and) Triethylhexyl Trimellitate (and) C30-45 Olefin | 1.50% |
| | (Clearwax, Sensient Cosmetic Technology - LCW) | |
| 12. | Octyldodecanol (Eutanol G, Cognis Corporation, Care Chemicals) | 18.50% |
| 13. | Isononyl Isononanoate (and) Polybutene (and) Pentaerythrityl Tetraisostearate (and) | 18.00% |
| | Isostearyl Alcohol (Covaclear, Sensient Cosmetic Technology - LCW) | |

Part D

| | | |
|---|---|---|
| 14. | Red iron oxide (AS 70421, Sensient Cosmetic Technology - LCW) | 5.00% |
| 15. | Yellow iron oxide (AS 70422, Sensient Cosmetic Technology - LCW) | 0.30% |
| 16. | Black iron oxide (AS 70423, Sensient Cosmetic Technology - LCW) | 0.30% |
| 17. | Titanium Dioxide (Unipure White LC 981 AS, Sensient Cosmetic Technology - | 6.30% |
| | LCW) | |

Part E

| | | |
|---|---|---|
| 18. | Silicone Vesicles of Example 9 | q.s. |

### Procedure

a. Disperse well the Iron Oxide (Unipure Red LC381) in the rest of phase A while stirring
b. Prepare phase B and pour phase B into phase A under stirring
c. Prepare phase D
d. Heat phase C to 60° C. until the waxes are melted
e. Add phase D to phase C
f. Heat AB to 50° C.
g. Add AB in CD (normally after complete removal of the bubbles) at 50° C.
h. Then let cool down to 40-45° C. and add part E
   Mix to homogeneous

1. A process for preparing a vesicle composition comprising:
I) combining;
A) an organopolysiloxane having at least one hydrophilic substituent group,
B) a water miscible solvent,
D′) a hydrophilic active,
to form a dispersion of the hydrophilic active,
II) combining;
A) an organopolysiloxane having at least one hydrophilic substituent group,
C) optionally, a silicone or organic oil,
D″) a hydrophobic active,
to form a dispersion of the hydrophobic active,
III) combining the dispersion of the hydrophilic active and the dispersion of the hydrophobic active and admixing water to form vesicles.

2. The process of claim 1 where step I or step II further comprises the addition of B′) a water miscible volatile solvent.

3. The process of claim 2 further comprising:
IV) removal of the water miscible volatile solvent.

4. The process of claim 1 wherein the organopolysiloxane is a silicone polyether having the formula: 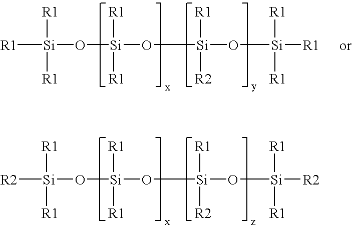
where R1 represents an alkyl group containing 1-6 carbon atoms;
R2 represents the group —(CH₂)ₐO(C₂H₄O)_{b}(C₃H₆₀)_{c}R3;
x is 1-1,000; y is 1-500; z is 1-500; a is 3-6; b is 4-20; c is 0-5;
and R3 is hydrogen, a methyl group, or an acyl group.

5. The process of claim 1 wherein the organopolysiloxane is a (AB)ₙ block silicone polyether having the formula;
—[R¹(R₂SiO))_{x′}(R₂SiR¹O)(CₘH₂ₘO)_{y′}]ₙ—
where x′ and y′ are greater than 4, m is from 2 to 4 inclusive, n is greater than 2.
R is independently a monovalent organic group containing 1 to 20 carbons,
R¹ is a divalent hydrocarbon containing 2 to 30 carbons.

6. The process of claim 1 wherein the water miscible solvent B) is a glycol.

7. The process of claim 6 wherein the glycol is propylene glycol.

8. The process of claim 1 wherein the water miscible volatile solvent B′) is an alcohol.

9. The process of claim 8 wherein the alcohol is ethanol or isopropanol.

10. The process of claim 1 wherein component C) is present and is a volatile methyl siloxane.

11. The process of claim 1 wherein D′) or D″) is a vitamin, sunscreen, fragrance, natural plant extract, or antioxidant.

12. The process of claim 1 wherein D″ is Vitamin A palmitate.

13. The process of claim 12 wherein D′ is Vitamin C.

14. A vesicle composition prepared according to the process of claims 1.

15. A personal care product comprising the vesicle composition of claim 14.

16. The personal care product of claim 13 wherein the personal care product is selected from an antiperspirant, deodorant, skin cream, skin care lotion, moisturizer, facial treatment, wrinkle remover, facial cleansers, bath oils, sunscreens, pre-shave, after-shave lotions, liquid soap, shaving soap, shaving lather, hair shampoo, hair conditioner, hair spray, mousse, permanent, hair cuticle coat, make-up, color cosmetic, foundation, blush, lipstick, lip balm, eyeliner, mascara, nail polishes, and powders.
